(19) United States
(12) Patent Application Publication
(10) Pub. No.: US 2023/0119502 A1
(43) Pub. Date: Apr. 20, 2023

(54) Collagen Nucleation Inhibitors

(71) Applicant: Northeastern University, Boston, MA (US)

(72) Inventor: Jeffrey PATEN, Mansfield, MA (US)

(21) Appl. No.: 17/915,638

(22) PCT Filed: Mar. 30, 2021
(86) PCT No.: PCT/US2021/025014

§ 371 Date: Sep. 29, 2022

Related U.S. Application Data
(60) Provisional application No. 63/002,273, filed on Mar. 30, 2020.

(51) Int. Cl. A61L 27/24; A61L 27/58; A61L 27/36; A61L 29/04

(52) U.S. Cl. CPC A61L 27/24; A61L 27/58; A61L 27/3645; A61L 29/045

(57) ABSTRACT

Solutions containing collagen monomers and saccharides inhibit the nucleation and growth of collagen fibrils. The solutions stabilize high concentrations of soluble collagen monomers, improving their incorporation into pre-existing tissues or collagen networks, thereby providing therapeutic applications of collagen for cosmetic treatments, wound healing, and injury repair involving damaged extracellular matrix. Pharmaceutical formulations, medical devices, and kits containing the solutions are provided.

### CROSS-REFERENCE TO RELATED APPLICATIONS

This application claims priority to U.S. Provisional Application No. 63/002,237, filed 30 Mar. 2020, which is incorporated by reference herein in its entirety.

### BACKGROUND

Collagen-based tissues (e.g., blood vessels, bones, cartilage, cornea, intervertebral discs, ligaments, tendons, sclera, and skin) are often subject to insufficient healing after injury. Causes may include inadequate collagen being produced by the cells at the injury site or difficulty in transporting available collagen molecules to the injury site or the collagen remaining soluble until reaching an injury site. A neutralized solution of collagen can spontaneously assemble in vitro into insoluble fibers under certain conditions, thereby complicating efforts to introduce collagen to promote injury repair. There is a need for therapies that utilize improved delivery of collagen to a site of injury to improve healing.

### SUMMARY

The present technology provides mechanisms to prevent a neutralized solution of collagen from spontaneously assembling into insoluble fibers if the collagen concentration is above 1 μg/mL. Because of the poor water solubility and low thermostability of collagen, the application of collagen is seriously limited in fields such as injectable biomaterials and cosmetics. However, the technology described herein prevents the spontaneous precipitation of collagen from high concentration solution, and the highly concentrated collagen solution does not disrupt or dissolve pre-existing tissues or collagen networks. Further, the solution-phase collagen herein can be readily incorporated into pre-existing tissues or collagen networks. The technology addresses the challenge of keeping therapeutic collagen soluble so it can diffuse to an injury site, incorporate, and repair, thereby opening up new possibilities for preventative and curative treatments, compositions, cell growth, and tissue synthesis, including 3D printing using the highly concentrated collagen solutions. The mechanisms to prevent a neutralized solution of collagen from spontaneously assembling into insoluble fibers can be in the form of collagen solutions that include only physiological molecules already present in the extracellular matrix of a subject, thereby providing a biocompatible, healing solution. Further, the concentrated collagen solutions can contain additional additives for specific applications designed to heal or strengthen extracellular matrix.

The present technology can be further summarized by the following list of features.

1. A method of stabilizing a collagen solution against polymerization, the method comprising incubating an aqueous solution comprising soluble collagen monomers at a concentration of at least about 1 μg/mL and one or more saccharides at a concentration of at least about 0.01 M; wherein polymerization of said collagen monomers and/or collagen fibril formation in the solution is inhibited.
2. The method of feature 1, wherein the one or more saccharides are selected from the group consisting of galactose, iduronic acid, glucuronic acid, N-acetyl-galactosamine, N-acetyl-glucosamine, and combinations thereof.
3. The method of feature 1 or feature 2, wherein the total concentration of the one or more saccharides is greater than about 0.01 M, or greater than about 0.1 M, or greater than about 0.2 M, or greater than about 0.5 M, or greater than about 1 M.
4. The method of any of the preceding features, wherein the solution comprises said collagen monomers at a concentration in the range from at least about 0.001 mg/mL to about 100 mg/mL.
5. The method of feature 4, wherein the collagen monomer concentration is at least about 0.5 mg/mL or at least about 1 mg/mL.
6. The method of any of the preceding features, wherein the pH of the solution is in the range from about 4 to about 10.
7. The method of any of the preceding features, wherein said incubating is carried out at a temperature in the range from about 0-40° C.
8. The method of any of the preceding features, wherein the solution further comprises a negatively charged ionic species.
9. The method of feature 8, wherein the negatively charged ionic species is selected from the group consisting of acetate, formate, citrate, lactate, a C₂-C₇ organic acid, or a combination thereof.
10. The method of any of the preceding features, wherein the solution further comprises one or more glycosaminoglycans selected from the group consisting of keratin sulfate, dermatan sulfate, heparin sulfate, chondroitin sulfate, hyaluronic acid, and combinations thereof.
11. The method of any of the preceding features, wherein the solution is devoid of any collagen solubility enhancers other than saccharides and glycosaminoglycans.
12. The method of any of the preceding features, wherein the solution further comprises collagen dimers, trimers, oligomers, aggregates, fibrils, or a combination thereof.
13. The method of any of the preceding features, wherein the solution is formulated for introduction into a body of a mammal, such as a human.
14. The method of any of the preceding features, wherein the collagen in said solution does not spontaneously polymerize or form collagen fibrils when stored for a period of at least about one month.
15. The method of any of the previous features, whereby nucleation of collagen polymerization is inhibited in the solution compared to a solution lacking said saccharide.
16. A method of promoting collagenous tissue repair and/or remodeling in a mammalian subject in need thereof, the method comprising the steps of:

(a) providing an aqueous repair solution comprising soluble collagen monomers at a concentration of at least about 1 μg/mL and one or more saccharides at a concentration of at least about 0.01 M; and

(b) contacting the repair solution with a tissue repair and/or remodeling site in the subject, whereby collagen monomers from the solution polymerize, or form collagen fibrils, or incorporate into existing collagen fibrils at said repair and/or remodeling site in the subject.

17. The method of feature 16, wherein the one or more saccharides are selected from the group consisting of galactose, iduronic acid, glucuronic acid, N-acetyl-galactosamine, N-acetyl-glucosamine, and combinations thereof.
18. The method of feature 16 or feature 17 wherein the total concentration of the one or more saccharides is greater than about 0.01M, or greater than about 0.1 M, or greater than about 0.2 M, or greater than about 0.5 M, or greater than about 1 M.
19. The method of any of features 16-18, wherein the repair solution comprises said collagen monomers at a concentration in the range from at least about 0.001 mg/mL to about 100 mg/mL.
20. The method of feature 19, wherein the collagen monomer concentration is at least about 0.5 mg/mL or at least about 1 mg/mL.
21. The method of any of features 16-20, wherein the pH of the repair solution is in the range from about 4 to about 10.
22. The method of any of features 16-21, wherein the repair solution further comprises a negatively charged ionic species.
23. The method of feature 22, wherein the negatively charged ionic species is selected from the group consisting of acetate, formate, citrate, lactate, a C₂-C₇ organic acid, or a combination thereof.
24. The method of any of features 16-23, wherein the repair solution further comprises one or more glycosaminoglycans selected from the group consisting of keratin sulfate, dermatan sulfate, heparin sulfate, chondroitin sulfate, hyaluronic acid, and combinations thereof.
25. The method of any of features 16-24, wherein the repair solution is devoid of any collagen solubility enhancers other than saccharides and glycosaminoglycans.
26. The method of any of features 16-25, wherein the solution further comprises collagen dimers, trimers, oligomers, aggregates, fibrils, or a combination thereof.
27. The method of any of features 16-26, which is performed in conjunction with a surgical procedure.
28. The method of any of features 16-27, wherein said contacting comprises injection, infusion, continuous infusion, diffusion, dermal application, spraying, or surgical application or implantation of the repair solution at said tissue repair and/or remodeling site.
29. The method of any of features 16-28, further comprising adjusting pH, ionic strength, collagen monomer concentration, and/or saccharide concentration of the repair solution.
30. The method of any of features 16-29, wherein the method speeds repair and/or healing of a damaged connective tissue at said tissue repair and/or remodeling site.
31. The method of any of features 16-30, wherein aids in treatment or repair in the subject of an injury, a wound, a bone fracture, a ruptured tendon, a ligament, a skin condition, or a damaged extracellular matrix.
32. The method of any of features 16-31, wherein the tissue repair site comprises a wound, a broken or fractured bone, a ruptured tendon, an injured ligament, a skin lesion, a scar, a hernia, a damaged barrier membrane, an eye or a portion of an eye, an inflammation of a connective tissue, a site suspected of being subject to future injury or tissue damage, or a site suspected of sustaining an injury.
33. A tissue remodeling solution for use in the method of any of features 16-32, the solution comprising soluble collagen monomers at a concentration of at least about 1 μg/mL and one or more saccharides at a concentration of at least about 0.01 M.
34. A medical device comprising the tissue remodeling solution of feature 31.
35. The medical device of feature 34 that is selected from the group consisting of a syringe, an implantable device, a wearable device, a wearable and removable device, an infusion device, a pump, and a catheter.
36. A tissue scaffold or artificial collagen-based tissue comprising the tissue remodeling solution of feature 33.
37. The tissue scaffold or artificial collagen-based tissue of feature 36 that is fabricated by a method comprising 3-D printing using the tissue remodeling solution of feature 33.
38. A kit comprising the device of feature 34 or 35 or the tissue scaffold or artificial collagen-based tissue of feature 36 or 37 and the tissue remodeling solution of feature 32.

As used herein, the term “biocompatible” refers to a material that is not harmful to living tissue. As used herein, a site at which such tissue remodeling or repair is targeted is referred to as a “tissue repair site”, which may include some surrounding tissue adjacent to an injury or target area. As used herein, a non-physiological molecule refers to a molecule of any size not typically found in a human or other mammalian subject, and a non-physiological environmental condition refers to a pH, temperature, or ionic strength or osmolality not typically found in a human or other mammalian subject.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the . Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1 shows a visual depiction of biological glycosaminoglycans (GAGs) including keratin sulfate (KS), dermatan sulfate (DS), heparin sulfate (HS), chondroitin sulfate (CS), and hyaluronic acid (HA). Their saccharide constituents, galactose, iduronic acid, glucuronic acid, N-acetyl-galactosamine, and N-acetyl-glucosamine, are reduced down to three unique molecules shown at center.

FIGS. 2A-2 C show effects of individual monosaccharides on 1 mg/mL collagen assembly in vitro. FIGS. 2D-2F show effects of individual monosaccharides on 0.5 mg/mL collagen assembly in vitro. Optical density (313nm, top, i) was measured in the presence of N-acetyl-glucosamine (GlcNAc, FIG. 2A, FIG. 2D), galactose (Gal, FIG. 2B, FIG. 2E), and glucuronic acid (GlucA, FIG. 2C, FIG. 2F). Kinetic data are extrapolated from the turbidity curves for GlcNAc (FIGS. 2A/2D, bottom, ii), Gal (FIGS. 2B/2E, bottom, ii), and GlucA (FIGS. 2C/2F, bottom, ii). Insets represent the normalized turbidity over 60 minutes. Normalized turbidity and kinetic data are not represented for conditions where collagen assembly was completely inhibited. Data represent the average of nine runs, and the kinetic data error bars represent the standard error of the mean.

FIGS. 3A-3B show collagen assembly in the presence of monosaccharides and acetic acid (CH₃COO⁻). FIG. 3A shows results of a turbidity assay to observe the influence of acetic acid on collagen assembly directly and in conjunction with the monosaccharide Gal. FIG. 3B shows kinetic data for comparison of select conditions. Error bars represent the standard error of the mean (n=9).

FIGS. 4A-4D show effects of monosaccharide combinations on 1 mg/mL collagen assembly. The optical density (top, i) associated with collagen fibrillogenesis was measured in response to the addition of 1:1 GlcNAc:Gal (FIG. 4A), 1:1 GlcNAc:GlucA (FIG. 4B), 2:1 GlcNAc:GlucA (FIG. 4C), and 1:2 GlcNAc:GlucA (FIG. 4D) at varying concentrations. The kinetic data (bottom, ii) were extrapolated from the turbidity curves. Insets depict the normalized turbidity for the first 60 minutes of the experiment. Normalized turbidity and kinetic data were only for conditions where collagen assembly was not completely inhibited. The data represents the average of 9 runs, and the kinetic data error bars represent the standard error of the mean.

FIG. 5 shows results for stability of pre-established collagen networks in the presence of saccharide solutions. The data for each condition (n=9) has been normalized to a 0-1 scale for comparison.

FIGS. 6A-6C show incorporation of collagen into a pre-existing network in the presence of critical monosaccharide concentrations. In each of FIGS. 6A-6C, the top row displays images of the pre-polymerized, fibrous A546-labeled collagen network. The middle row captures the same location but identifies the A488-labeled collagen that is added as mixture with the monosaccharides. The bottom row shows the overlay product of the two isolated fluorophore images. The data is acquired in the presence of either 0.5 M GlcNAc (FIG. 6A), 0.5 M Gal (FIG. 6B), or 1 M GlucA (FIG. 6C).

FIGS. 7A-7B show representative microscope images of collagen network before and after exposure to Gal. A reverse turbidity experiment was performed with an inverted microscope to observe changes in the collagen network at 37° C. for 180 minutes. The images illustrate minimal to no change in the network after exposure to Gal. The images are representative of all conditions.

FIG. 8 shows a reverse turbidity plot for 1 mg/mL collagen network with 0.5 M Gal with 0.5 M acetic acid, along with a 1XTBS control plot. The change in optical density of a pre-established collagen matrix in the presence of a solution of 0.5 M Gal with 0.5 M acetic acid was tracked for 6 hours at 313 nm at 37° C.

### DETAILED DESCRIPTION

The present technology provides therapeutic compositions and treatments for biological tissues, particularly for the extracellular matrix of collagen-containing tissues. The technology provides methods to prevent a neutralized solution of collagen from spontaneously assembling into insoluble fibers if the concentration is above 1 μg/mL. Concentrated solutions of collagen can be prepared and administered to tissues or collagen networks, without the collagen spontaneously coming out of solution. While spontaneous precipitation of collagen is prevented, the solution-phase collagen can be readily incorporated into pre-existing tissues or collagen networks without disruption of the pre-exiting structures. The technology addresses the challenge of keeping therapeutic collagen soluble so it can diffuse to an injury site, incorporate, and repair.

Previous collagen therapies have existed as patches of collagen or sponges to address the burden of providing the low-concentration collagen protein to the biological system. These techniques can lead to disruption of the existing tissues or collagen networks. Without something to stabilize the soluble collagen, there is a high risk of fibrosis and formation of excess tissues that inhibits native function of the tissue being treated or causes secondary problems. The present technology overcomes the problem of spontaneous assembly of collagen from solution (e.g., stabilization of the soluble collagen), leading to a variety of delivery options for high concentration collagen solutions, in addition to patches or sponges. Examples of treatments provided herein include preventative wound care for collagen-based tissues, accelerated wound care for collagen-based tissues, and treating acute or chronic collagen-based pathologies (e.g., tendinitis). A therapeutic treatment for injuries herein can contain only naturally occurring biological molecules, match the physiological environment, and deliver at a concentration which reduces volumes from liters to milliliters. Alternatively, additives can be included in the solutions herein that are non-physiological as needed, for example, to further speed the incorporation or binding of the solution-phase collagen to the pre-existing tissues or collagen network. The technology provides monosaccharide-water interactions that alter the nucleation and growth of collagen fibrils. The individual saccharides of glycosaminoglycans (biologically produced chains of saccharides) have the capacity to raise the critical concentration at which collagen spontaneously assembles by about 3-5 orders of magnitude. Using the technology described herein, for example, 1-100 milligrams of collagen protein can be delivered within 1 milliliter of solution, instead of the previously required 1-100 liters of solution. The collagen can remain soluble at 3-5 orders of magnitude greater than the concentration previously possible in vitro without the need for any non-physiological molecules or environmental conditions. The mixtures of collagen and saccharides do not disrupt or destroy pre-existing tissues. The mixture of collagen and saccharides permits the soluble collagen to incorporate into pre-existing tissues even though the collagen in solution does not spontaneously polymerize. Using the hydrogen bonding strategies disclosed herein, disaccharides, oligosaccharides, shortened sections (fragments) of naturally occurring glycosaminoglycans, and the intact, full naturally occurring forms of glycosaminoglycans can operate in the same manner as the monosaccharides to prevent collagen polymerization. The individual saccharides, oligosaccharides, fragments of glycosaminoglycans, intact glycosaminoglycans, and any of the forementioned items can be further attached to a protein core (i.e., a full or partial version of a naturally occurring proteoglycan core) to provide the stabilized high concentration collagen solutions.

Collagen serves as the premier, load-bearing molecule in vertebrates and is responsible for sustaining a myriad of forces applied to the human musculoskeletal system. Tissue strength is conferred from densely packed bundles of fibrils that run continuously throughout and orient with precision to best resist tension, compression, torsion, and pressure (Hijazi, K. M., et al., 2019). Shortly after cells establish the initial template for their designated tissue, they transition from a high motility state to a relatively static state (Kohler, J., et al., 2013). Serving now as contractile units, mechanical detectors, and molecular secretors, the cell population can no longer maintain the dense extracellular matrix (ECM) by migration and direct deposition, as observed during tissue genesis (Birk & Trelstad, 1986; Richardon, S. H., et al., 2007). Even at the early neonatal stage, the collagen fibrils have become restrictively dense by occupying an area fraction of over 75%, the cell population has diminished to a volumetric fraction of approximately 5%, and nearest cell neighbors are tens of microns away (Moore & De Beaux, 1987; McBride, D. J., et al., 1985; Kalson, N. S., et al., 2015; Nagy, I. Z., et al., 1969). With increasing age, collagenous tissues experience a further decline in cell density, nutritional supply, and cell metabolism (Tuite, D. J., et al., 1997).

An imbalance in the accrual rate of damage as compared to the repair rate is principally onset by the decreased capacity of the interstitial fluid to stabilize the monomeric (i.e., unassembled) phase of collagen. Collagen solubility is of critical importance because cells are largely immobilized within the dense network of fibrils and rely on diffusive transport of proteins and enzymes to maintain the ECM. In young tissues, the interstitial fluid contains a high concentration of glycosaminoglycans (GAGs), which are negatively charged polysaccharide chains with a high propensity to bind water (Aukland & Nicolaysen, 1981; Aukland & Reed, 1993). With increasing age, dwindling cell populations fail to meet the demand of GAG production (Riley, G. P., et al., 1994; Ippolito, E., et al., 1980). While GAGs have a plethora of other known biological roles (Ryan, C. N., et al., 2015) data suggests novel functionality whereby GAGs make collagen nucleation thermodynamically unfavorable and thus stable as soluble individual molecules. The GAG stabilization of collagen can raise solubility by about three or more orders of magnitude and allows the ECM to be bathed in interstitial fluid containing rich concentrations of collagen for rapid repair potential.

Given that GAGs are either commercially unavailable or prohibitively expensive, the technology can more easily be applied with the individual subunits of the biological GAGs. The individual subunits of the biological GAGs are utilized to demonstrate the effectiveness of the technology and the mechanisms of the technology. The individual subunits, disaccharides, fragmented GAGs, or native GAGs can be utilized to increase the solubility limit (e.g., the concentration that a solute precipitates out of solution) of collagen, taking into consideration the upper solubility of the GAGs or fragments thereof, as well in incubation with a collagen monomer solution. There are four GAG chains commonly found throughout the body's ECM and interstitial fluid: chondroitin sulfate, dermatan sulfate, heparan sulfate, and keratan sulfate. Each of these are chains including smaller subunits called saccharides, and examples are provided in Table 1:

TABLE 1
Examples of GAG Chains and Component Saccharides

| GAG Chains | Component Saccharides |
|---|---|
| Chondroitin sulfate, CS | glucuronic acid and N-acetyl-galactosamine |
| Dermatan sulfate, DS | iduronic acid and N-acetyl-galactosamine |
| Heparan sulfate, HS | glucuronic acid, iduronic acid, and N-acetyl- |
| | glucosamine |
| Keratan sulfate, KS | galactose and N-acetyl-glucosamine |

In Table 1, the four GAG chains provide a corresponding list of five relevant saccharides. Glucuronic acid and iduronic acid are isoforms of the same molecule. Similarly, N-acetyl-galactosamine and N-acetyl-glucosamine are isoforms of the same molecule. This reduces the list of unique saccharides needed to replicate the functionality of all the GAGs in the human body down to three molecules: glucuronic acid, N-acetyl-glucosamine, and galactose. Herein, the term isoform, when referring to a single monomer, molecule, or saccharide (e.g., glucuronic acid and iduronic acid) can be utilized to refer to a diastereomer or enantiomer. When referring to longer sequences of monomers, molecules, or saccharides, the term isoform can also be utilized to refer to two or more functionally similar sequences (chains) of monomers that have a similar but not identical sequences (chains) and are either modified differently during in vivo assembly and/or encoded by different genes or by RNA transcripts from the same gene which have had different exons removed.

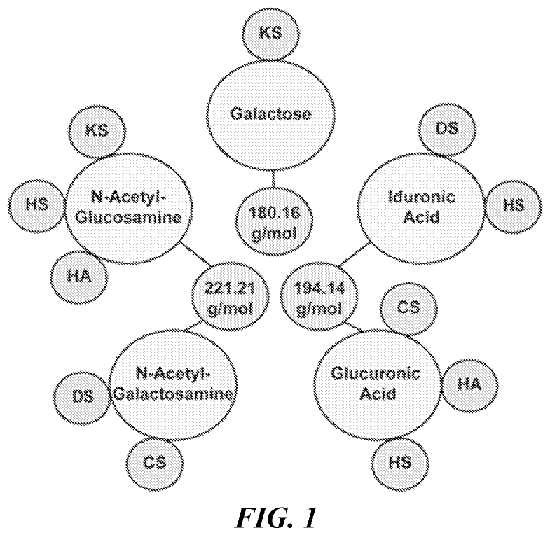

A visual depiction of biological glycosaminoglycans (GAGs) including keratin sulfate (KS), dermatan sulfate (DS), heparin sulfate (HS), chondroitin sulfate (CS), and hyaluronic acid (HA) is shown in FIG. 1. Their saccharide constituents, galactose, iduronic acid, glucuronic acid, N-acetyl-galactosamine, and N-acetyl-glucosamine, are reduced down to three unique molecules shown at the center of FIG. 1 as 180.16 g/mol, 221.21 g/mol, and 194.14 g/mol. Only focusing on individual diastereomers, for example, in FIG. 1, the 194.14 g/mol can represent the molecular weight of glucuronic acid, the 221.21 g/mol can represent the molecular weight of N-acetyl-glucosamine, and the 180.16 g/mol can represent the molecular weight of galactose.

Herein each of these three saccharides demonstrates the ability to inhibit nucleation and formation of new collagen structures, even with collagen concentration at or greater than 1000 times the previously considered threshold concentration of 1 μg/m L. Under the conditions disclosed herein, the collagen is still functionally capable of interacting with pre-existing matrices, such as damaged tissues, and the saccharides do not disrupt the pre-existing tissues.

Proteoglycans (PGs) are ubiquitously present throughout all tissues in the human body. They are classified as either intracellular, cell surface-bound, pericellular, or as part of the extracellular matrix (ECM) and are further organized by genetic homology (lozzo & Schaefer, 2015). Of the 44 distinct genes that encode for the protein core of PGs, 18 of those belong to the small leucine-rich proteoglycans (SLRPs) found in the ECM (Schaefer & lozzo, 2008). In addition to the protein core, SLRPs often contain one or more covalently attached glycosaminoglycan (GAG) chains via a linker tetrasaccharide (Silbert & Sugumaran, 2002; Sugahara & Kitagawa, 2002). The protein core typically ranges between about 25-65 kDa and accounts for less than 50% the total molecular weight (Brown, S., et al., 2012; Hassell, J. R., et al., 1986). The GAG chains are negatively charged, linear polymers of repeating disaccharide units that alternate between amino sugars and either uronic acids or galactose residues. The glycan composition, chain length, sulfation pattern, and epimerization are highly heterogenous due to regulation in the Golgi apparatus rather than being directly encoded by the genome (Mende, M., et al., 2016; Prydz & Dalen, 2000. This produces GAGs which vary spatially, temporally, and across a single cell type (Caniggia, I., et al., 1992). The molecular diversity of PGs is associated with many functions and interactions (lozzo & Schaefer, 2015). While the molecular diversity of PGs yields an exquisitely long list of functions and interactions (lozzo & Schaefer, 2015), the heterogeneity and polydispersity often obfuscate the exact role of a PG across multiple studies (Li, L., et al., 2012).

One of the better studied protein-PG systems is between type I collagen (collagen) and the SLRPs that contain GAG chains of either chondroitin sulfate (CS), dermatan sulfate (DS), or keratan sulfate (KS). Despite considerable research into SLRP-collagen relationships (Chen & Birk, 2013), their exact interactions are complicated by factors such as overlapping binding sites. As determined from the presence of CS or DS chains, multiple PGs selectively bind to the d and e sub-bands within the gap/overlap region of collagen fibrils, and similarly those PGs with KS chains preferentially bind to the a and c sub-bands (Scott, J. E., 1988; Miyagawa, A., et al., 2001; Pringle & Dodd, 1990). Further complexity arises from the temporally changing expression levels of PGs and GAGs. For instance, biglycan is highly expressed in earlier stages of corneal development (Zhang, G., et al., 2009), intervertebral disc development (Melrose, J., et al., 2001), and tendon wound healing (Dunkman, A. A., et al., 2014), but its presence progressively declines in favor of decorin. Similarly, fibromodulin stabilizes early-stage collagen fibril intermediates, and lumican serves to regulate lateral fibril growth in the later stages (Chakravarti, S., 2002). While exact functions are often difficult to discern due to spatial overlap, temporal shifts, and partial compensation between PGs, knockout studies have revealed a broad range of phenotypes. SLRPs alter assembly kinetics (Vogel, K. G., et al., 1984; Chakravarti, S., et al., 2006), tissue hydration (Liu, C. Y., et al., 2003; Chakravarti, S., et al., 2000), and structural mechanics (Chakravarti, S., et al., 1998; Ameye, L., et al., 2002; Fust, A., et al., 2005), while also regulating fibril diameter (Ameye, L., et al., 2002; Zhang, G., et al., 2006), interfibrillar spacing (Chakravarti, S., et al., 1998; Corsi, A., et al., 2002), and enzyme activity of both lysyl oxidase (Kalamajski, S., et al., 2016; Maruhashi, T., et al., 2010) and matrix metalloproteinases (Geng, Y., et al., 2006; Pietraszek, K., et al., 2014).

The multitude of physiological roles attained by PGs are primarily attributable to either steric hindrance or the hygroscopic nature of the GAG chains (Kalamajski & Oldberg, 2010; Han, E. H., et al., 2011). The constituent monosaccharides consist of multiple polar functional groups that interact with surrounding water molecules via an electrostatic attraction between the negatively charged carboxylate groups or sulfate groups and the hydrogen of the water molecules (Ruiz Hernandez, S. E., et al., 2015). Additionally, monosaccharides contain multiple hydroxyl groups that are capable of hydrogen bonding as both a donor and acceptor site, for example, as shown in Scheme 1 (Harvey & Symons, 1978; Petukhov, M., et al., 2004). The hydrogen bonding between GAGs and water produces strong translational and orientational ordering of the solvent, which raises the overall free energy of the microenvironment (Huggins, D. J., 2015; Li & Lazaridis, 2003). Collagen fibrillogenesis is an endothermic process whose spontaneity relies on entropic changes in the surrounding solvent (Cooper, A., 1970).

Collagen assembly in vitro is spontaneous down to a threshold concentration of approximately 1 μg/mL; (Li, S., et al., 2003; Kadler, K. E., et al., 1987) however, this is rather unlikely to be the threshold in vivo due to the influence of the about 200 glycoproteins in the matrisome (Hynes & Naba, 2012). Additionally, multiple studies point towards a dependence on both protein and cell interactions in vivo (Paten, J. A., et al., 2019 and references therein).

To understand the role that saccharides play in the GAG structure and collagen assembly, the effects of individual monosaccharides on collagen nucleation and incorporation are explored, while removing the complexities introduced by variable GAG length, composition, and degree of sulfation. Monosaccharides that constitute GAG chains involved in protein-GAG complexes in the ECM are selected and studied. Similarities in the molecular composition of GAGs is illustrated, for example, in FIG. 1. As shown in FIG. 1, five saccharides support the formation of six GAG chains. FIG. 1 shows a visual depiction of the biological GAGs, their saccharide constituents, and the reduction down to three unique molecules (center), whereas two sets of the monosaccharides are stereoisomers. Heparan sulfate and hyaluronic acid fall outside of the SLRP family but have similar molecular composition to the GAGs of SLRPs.

The list of unique saccharides is further reduced to three, as N-acetyl-glucosamine and N-acetyl-galactosamine are isoforms, as are D-glucuronic acid and L-iduronic acid. To minimize redundancy, the mechanisms are explained with the N-acetyl-glucosamine (GlcNAc), galactose (Gal), and glucuronic acid (GlucA), with structures represented in Scheme 1.

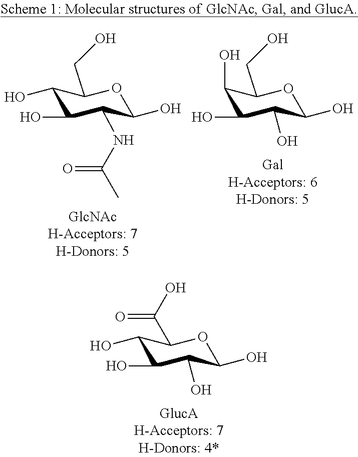

A threshold collagen concentration is determined in the presence of each saccharide. Further, a 1:1 ratio of GlcNAc:Gal, to replicate the composition of KS and GlcNAc:GlucA, to replicate the composition of CS, DS is investigated, and hyaluronic acid (HA). The effect of these saccharide solutions on pre-existing collagen matrices is also investigated. Finally, the ability of collagen to incorporate into pre-established matrices in the presence of saccharides is investigated. The data herein indicate that monosaccharides can raise the threshold concentration by about 3-5 orders of magnitude without disrupting collagen's ability to incorporate into pre-established matrices, networks, and tissues.

For the three monosaccharides in Scheme 1, the number of hydrogen bond acceptor (H-acceptor) and hydrogen bond donor (H-donor) sites are listed for each molecule. Notably, the functional group in GlucA is the carboxylic acid, which was deprotonated during the studies herein, due to a 3.2 pKa value. The asterisk (4*) above the GlucA chemical structure indicates that the number of hydrogen-donor sites was calculated with the deprotonated form of the carboxylic acid. When determining the number of H-acceptors and H-donors on a chemical structure to be utilized to enhance collagen solubility, attached sulfates (e.g., —SO₂OH) should be similarly considered as deprotonated. Sulfur in a thiol or an ether will be counted as an H-acceptor, unless other nearby substituents or C═C moieties change the pKa values.

When the saccharides are in GAG chains, there is a potential for a multifaceted regulatory system. As collagenous tissues transition from embryonic development to maturity, the tissue composition shifts dramatically from a predominantly cellular volume to a restrictively dense network of fibrillar arrays (Kaye, G. I., 1969; Hayes, A. J., et al., 2001; Hunziker, E. B., et al., 2002; Boos, N., et al., 2002; Moore & De Beaux, 1987; McBride, D. J., et al., 1985; Kalson, N. S., et al., 2015; Nagy, I. Z., et al., 1969). Priority is given to load bearing structures, and ECM micro-environments of fiber bundles are established with the nearest cells often residing tens of microns away. Despite this restricted cellular access, the collagen fibrils are still reliant on constant self-repair (Ker, R. F., et al., 2000). This point is supported by the work of Wang and Ker, where they show excised tendon failure after about 28 hours when cyclically stretched at 1 Hz to the physiological stress level of normal locomotion (Wang & Ker, 1995; Wang, X. T., et al., 1995). It is believed that tissue maintenance relies heavily on the rich molecular milieu of the interstitial fluid surrounding the ECM. However, it remains unclear whether the interstitial fluid can support appreciable concentrations of collagen necessary for tissue repair, given in vitro observations of spontaneous collagen assembly above a 1 μg/mL threshold (Li, S., et al., 2003; Kadler, K. E., et al., 1987). It is believed that GAGs, specifically their monosaccharide constituents, play an important role in stabilizing monomeric collagen in interstitial fluid is motivated in part by the correlation between the decreased capacity for wound healing and the decreased GAG quantity, chain length, and negative fixed charge density associated with aging (Gould, L., et al., 2015; Li, Y., et al., 2013; Ng, L., et al., 2003; Grande-Allen, K. J., et al., 2004; Lee, H. Y., et al., 2013). Their ubiquity as both surface-bound and soluble agents potentiates a multifaceted regulatory system that controls tissue inter/intrafibrillar interstitial fluid volume, the abundance/access to reparatory molecules, and the ECM site accessibility for integration.

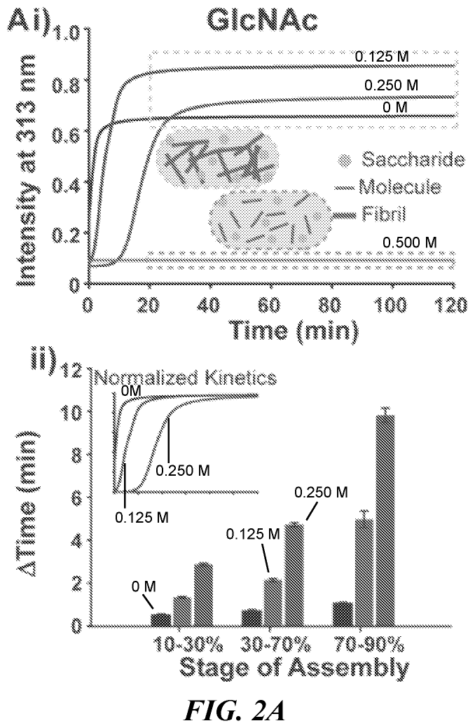

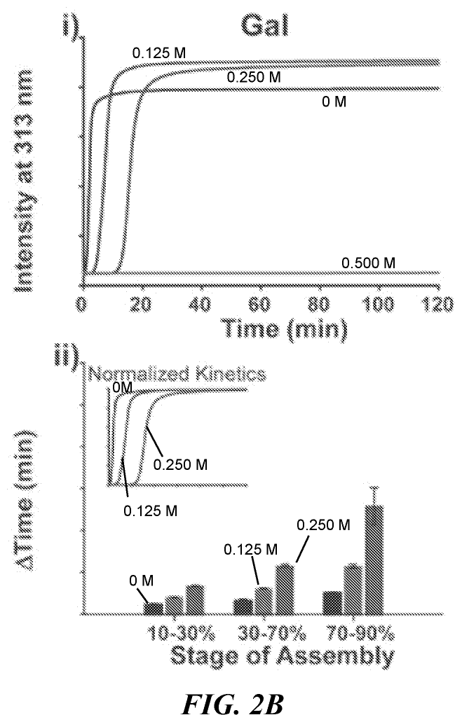

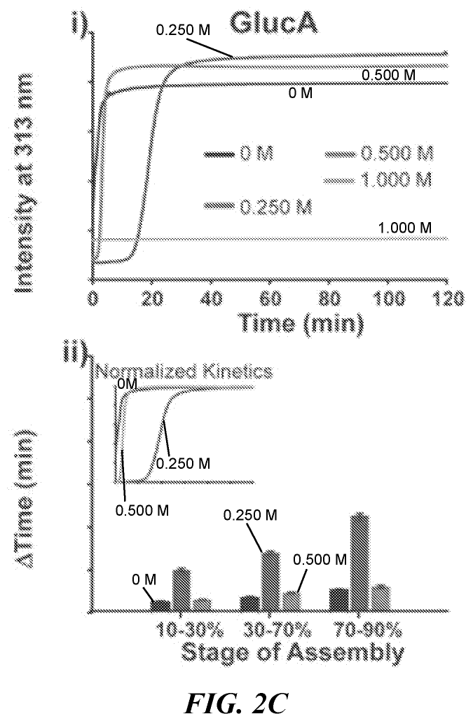

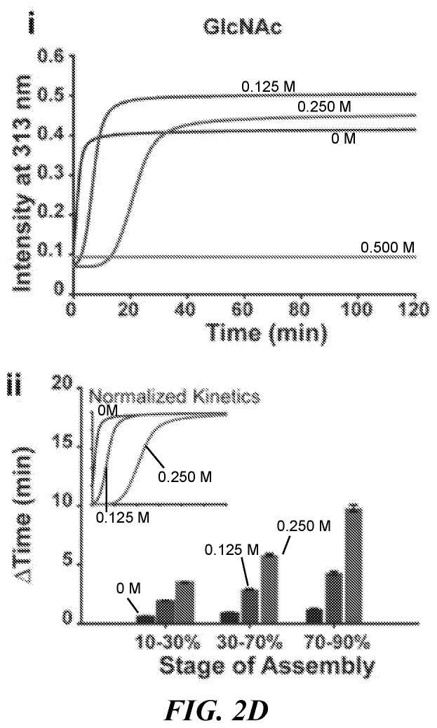

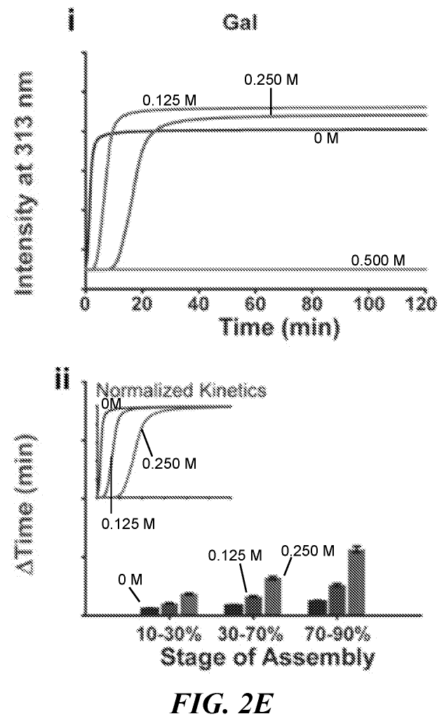

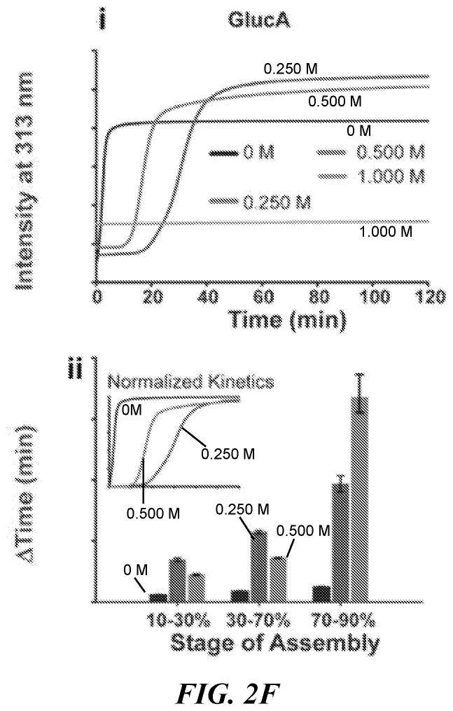

The naturally hygroscopic monosaccharides undergo preferential hydrogen bonding with the bulk water to reduce the overall entropic state of the solvent. In this state, a critical concentration can be considered to be that which makes it thermodynamically unfavorable for collagen-bound water to dissociate; thus, there is no longer the entropy gain required for spontaneous assembly of collagen. In FIGS. 2A-2C, the optical density associated with collagen fibrillogenesis (intensity at 313 nm) is shown versus time for a 1 mg/mL collagen solution including GlcNAc (FIG. 2A), Gal (FIG. 2B), or GlucA (FIG. 2C). A 1 mg/mL collagen solution control solution, without saccharide, is also shown. In FIGS. 2D-2F, the optical density is shown versus time for a 0.5 mg/mL collagen solution including GlcNAc (FIG. 2D), Gal (FIG. 2E), or GlucA (FIG. 2F). A 0.5 mg/mL collagen control solution is shown. The effect that these monosaccharides have on inhibiting collagen assembly is independent of collagen concentration. The reliance on saccharide interactions with water rather than with the protein is supported by the finding that the same critical concentrations of monosaccharides inhibit nucleation for both 0.5 and 1 mg/mL collagen.

When other inhibitors such as disaccharides, oligosaccharides, fragments of glycosaminoglycans, intact glycosaminoglycans, and any of these attached to a protein core (i.e., a full or partial proteoglycan), or mixtures thereof, are utilized to inhibit nucleation of collagen, considerations are the solubility of each inhibitor and the interactions with water to form a solvent cage (ordering the bulk water) including water hydrogen-bound to the inhibitors (e.g., water, hydrogen-bound to —OH groups on the inhibitors). When water forms hydrogen bonds with the inhibitors, a solvent cage is formed, ordering the bulk water, and the —OH groups or other similar hydrogen bond acceptors (H-acceptors) are considered “structure-makers”. For example, the solvent cage can be considered a structure formed with surrounded water around the solute (“structure maker”). The solute can then be described as an encapsulated particle, and while in solution the solute and the solvent cage form conformations best described and modeled with a Boltzmann weighted distribution of the conformations. When the solution (including the water and solute) is frozen, both the solvent cage and the solute are frozen into a single conformation, which in the solid state can be described as a frozen conformation, crystal, cocrystal, interspersed polycrystalline, or combination structure, no longer in a Boltzmann weighted distribution of conformations but frozen into one conformation. The working ranges for the collagen inhibitors disclosed herein are about 0° C.-40° C. At higher temperatures, not only do the number of conformations of the solute and solvent cage increase, but H-bonds can also be broken.

Similarly, Cooper calculated the free energy changes associated with the addition of ions and small molecules and concluded that each solute is either a “structure-maker” or a “structure-breaker” with water, where structure-makers increase the free energy, and structure-breakers decrease the free energy (Cooper, A., 1970). Both computational and experimental studies identify strong hydrogen bonding between the hydroxyls present on monosaccharides and bulk water, further supporting the conclusion that the GAG monosaccharides are structure-makers with water (Ruiz Hernandez, S. E., et al., 2015; Harvey & Symons, 1978). While longer saccharides and the intact GAGs are also structure makers and can be utilized herein, the solubility of the longer saccharides and intact GAGs can be less than the GAG monosaccharides. Simultaneously, the number of H-acceptors and H-donors on a larger saccharide and intact GAG is significantly more than on a monosaccharide, so with complete solubility of the inhibitor, the total concentration (e.g., molarity) of structure-makers for a given larger saccharide and intact GAG is higher. For example, 1 M of a given disaccharide can provide almost double the concentration of H-acceptors compared to 1 M of the saccharide. In this example, the loss or gain of H-acceptors due to the bonding between disaccharides must be considered. Similarly, for longer saccharide chains, the bonding between saccharides is considered.

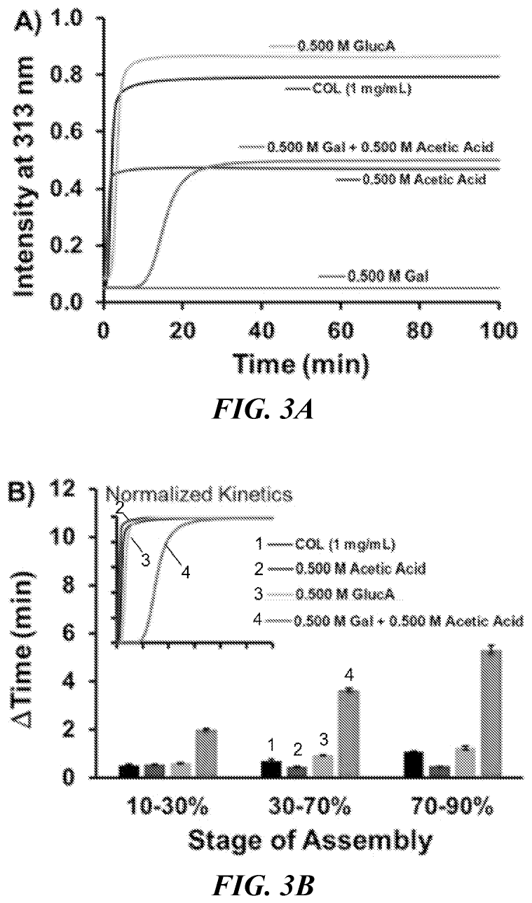

Upon further examination, the hydrogen bonding mechanism can explain why different monosaccharides have different critical concentrations to suppress collagen assembly. For example, GlucA requires a higher critical concentration to suppress collagen assembly, as is shown in FIG. 2C, likely due to a balance between structure-making hydroxyl groups and potentially a structure-breaking carboxylate group. Through molecular simulations, Markham et al. revealed that the interactions between the carboxylate functional group in an acetate anion and water would lead to an increase the entropy of the system (Markham, G. D., et al., 1997). This finding supports the data herein, where a greater concentration of GlucA is required due to the compromised net effectiveness at ordering the bulk water and increasing the free energy. The addition of acetic acid (i.e., a carboxylate group) to Gal, without covalently attaching the carboxylate to Gal, replicates the effects observed in the GlucA test condition. The addition of acetic acid to Gal hinders Gal's capacity to completely inhibit collagen nucleation. In FIG. 3A, a 1 mg/mL collagen solution including 0.5 M Gal and 0.5 M acetic acid show that the addition of acetic acid replicates the 0.5 M GlucA condition. This further indicates that the carboxylate group of GlucA causes a structure-breaking increase in the entropy of the system, viewed as a system of structure-making hydroxyl groups, which order the water, with the structure-breaking carboxylate.

In aqueous solution, in the range of pH from about 4-10, spontaneous collagen assembly can be prevented at concentrations of collagen greater than 1 μg/mL by incubating the collagen solution in a saccharide, combination of saccharides, or longer chains thereof in the range up to about 1 M or higher. The lower concentration limit is about 0.01 M. The upper concentration limit can be higher than 1 M, and this limit can be determined by, for example, the maximum solubility of the saccharide, disaccharide, oligosaccharide, GAG fragment, GAG, or the physiological conditions planned. The saccharide (or chain) can be selected based upon the number of hydrogen-donor sites and hydrogen acceptor sites. As discussed above, the pKa values of functional groups should be taken into consideration. Various functional groups, for example amides, hydroxyls, sulfation, carboxyl, sulfates (e.g., —SO₂OH), or carboxylic groups, can be attached or added to a saccharide or longer chain to tailor the number of H-donors and H-acceptors for various applications.

When inhibitors such as water-soluble saccharides, disaccharides or oligosaccharides, GAG fragments, or full GAGs are applied, the required inhibitory concentration can be calculated based upon the total number of H-donors and H-acceptors available on the specific inhibitor. For example, the concentration of H-donors, the concentration H-acceptors, or a ratio of these concentrations can be applied for a prediction of the critical concentration to suppress spontaneous collagen assembly. Additional additives, for example organic acids (e.g., acetate, formate, citrate, lactate, a C₂-C₇ organic acid, or a combination), can also be utilized for structure breaking effects as in the example of acetic acid added to Gal (FIG. 3A). When these structure breaking additives are used not covalently attached to the inhibitor, the effects upon kinetics of the subsequent collagen assembly inhibition will not match those of when the structure breaking additive (or functional group) is directly attached to the inhibitor, as is the case with GlucA with its attached carboxylic acid. These effects can be described by examining the cage effect, or ordering of water, discussed above. When the carboxylic acid (or other acid) functional group is attached to the collagen inhibitor, the ordering of water or structure maker ability of the inhibitor is weakened. The weakening can be thought of as a hydrogen-bound water solvent cage disrupted by the (deprotonated) carboxylic acid. When the carboxylic acid (or other acid) is not attached to the collagen inhibitor, the structure maker ability, or the ordering of water surrounding the inhibitor, is not affected by the solute (which is internal to the surrounding ordered water), but instead is affected by the acid external to the solute and its associated solvent cage. Additional varieties of functional groups, for example, different degrees of sulfation introduced to modify the saccharides, chains, or GAGs, can be covalently attached and will have effects as illustrated by the effects observed for GlucA.

For in vitro applications, for example, 3D-printing of collagen networks, inorganic acids or otherwise non-physiological additives can be further utilized to quickly induce collagen fibril formation. For example, an additive is added during 3D-printing to induce a concentrated solution of collagen to polymerize at the exact spot it is printed at. The technology provides a highly concentrated collagen solution for application during 3D-printing and a method to induce polymerization of the highly concentrated solution after it is applied. Strain can also be applied to the concentrated collagen solution to induce fibril formation.

A printing ink can be formulated from the highly concentrated collagen solutions disclosed herein. Depending on the type of collagen, the ink can resemble a hydrogel or gel at collagen concentrations greater than about 25 mg/mL, greater than about 50 mg/m L, or greater than about 75 mg/mL. The ink can be used for 3D-printing external to a surgical site or a tissue repair site, for example, to construct a tissue scaffold. The ink can be utilized at a surgical site or a tissue repair site, for example, by fixating a knee, shoulder, or angle of a subject and utilizing a 3D-printer, surgical robot, or jet printer to apply the solution to accurate locations within the surgical site or tissue repair site. Particularly of interest is reconstruction of skin, for example, for burn patients or for patients requiring reconstruction of a scar area. Photon or two photon crosslinking can be used after printing. A post-printing crosslinking or polymerization of the collagen solution can be utilized, for example, by a radical polymerization-mediated gelation. Visible light can be utilized as a crosslink-mediator with the photo-excitation of tris(2,2′-bipyridyl)dichlororuthenium(II) hexahydrate (Ru) which is modified from RU²⁺ to Ru³⁺ through electron donation to sodium persulfate (SPS), which dissociates into sulfate anions causing covalent bonds between methacryloyl groups. Polymerization of the collagen monomers can be induced, for example, through stress, application of high frequency sound, spray of an organic acid, dehydration, or addition of a scaffold fiber upon the printed solution.

3D-printing of fibrillar collagen architectures can be accomplished wherein the stresses and strains associated with the printing process override the inhibitory effect of the saccharides and initiate the collagen assembly.

Live cells can be included in the liquid collagen compositions disclosed herein. In this example, the printing application process should not include shear or stress that disrupts the living cells. Cells can be isolated from donor tissue, including from a subject intended to receive the composition, optionally encapsulated within cytocompatible polymeric matrices and printed at a resolution that matches the heterogenic components of natural tissue, down to the microscale.

The concentration of the collagen solutions can be in the range from about 1 mg/ml to about 10 mg/mL, in the range from about 10 mg/mL to about 20 mg/mL, in the range from about 20 mg/mL to about 30 mg/mL, in the range from about 30 mg/mL to about 40 mg/mL, in the range from about 40 mg/mL to about 50 mg/mL, in the range from about 50 mg/mL to about 60 mg/mL, in the range from about 60 mg/mL to about 70 mg/mL, in the range from about 70 mg/mL to about 80 mg/mL, in the range from about 80 mg/mL to about 90 mg/mL, or in the range from about 90 mg/mL to about 100 mg/mL. Surgical implants can be constructed either by 3D-printing, molding, or injection of the materials disclosed herein. Sheets of the highly concentrated collagen solutions can be formed. The sheets can form collagen fibrils and can be utilized for grafting, implantation, or reconstruction. For example, the formation of collagen sheets (grafts, patches, layered for surrogate tissue) through the application of mechanical initiation (shear or extensional strain) to collagen/saccharide solution can be enabled by the methods and compositions disclosed herein.

The collagen solutions disclosed herein can be readily incorporated into pre-existing tissues without disruption of the collagen network or tissue. It is contemplated that the aqueous solutions disclosed herein can be applied in almost any pharmaceutical or medical formulations, for example, lipid nanoparticles, gels, capsules, infusions, intravenous (IV), injections, implants, nanocapsules, lyophilized (ready to reconstitute) formulations, implantable formulations, time release formulations, and patches; and it is expected that stability studies, crystal studies, cocrystal studies, dissolution studies, toxicity studies, etc. can all be accomplished for various formulations as is known in the industry. In an example, the technology can be provided in a dry mix form, ready to reconstitute before use, optionally in a kit.

Various visualization ingredients, for example, radioisotopes, stains, or fluorescent imaging can be utilized to monitor the effectiveness of the collagen formulations herein by a medical professional, with no disruption of pre-existing tissues. The technology also contemplates the use of nanoparticles for a variety of applications, for example, to induce a concentrated collagen solution provided herein to form fibrils at an exact location.

With the nontoxicity of the ingredients provided (i.e., ingredients already present in the ECM), it is expected that biocompatibility of the technology provides a huge opportunity for a variety of applications. In some instances, non-physiological additives can be utilized in medical and/or pharmaceutical applications, for example, to tailor delivery, to increase incorporation or binding, or to preserve the solutions for longer-term delivery or storage. Further, the technology can be formulated in a large variety of cosmetics, pet care products, cell culture kits, fabrication devices, and in industrial production as the envisioned applications are developed.

It is known that collagen is the most abundant protein in mammals, many types of collagen (about 28 types) have been discovered, and it is expected that more types will be discovered. The mechanisms disclosed herein are expected to work for all types of fibrillar collagen (e.g., I, II, III, V, XI) either presently known or unknown. It is further thought the technology can be applied in almost any area of a subject, including external application or application in highly sensitive areas (e.g., brain tissue or heart tissue). The technology includes applications for cartilage, intervertebral disks, and skin. Other extracellular matrix molecules, or agents acting on such molecules, can be added to the collagen/inhibitor solution (e.g., one or more of collagen type II, collagen type III, elastin, fibronectin, hydroxyapatite, lysyl oxidase, collagenase, matrix metalloproteinase, growth factors, and anti-inflammatory agents).

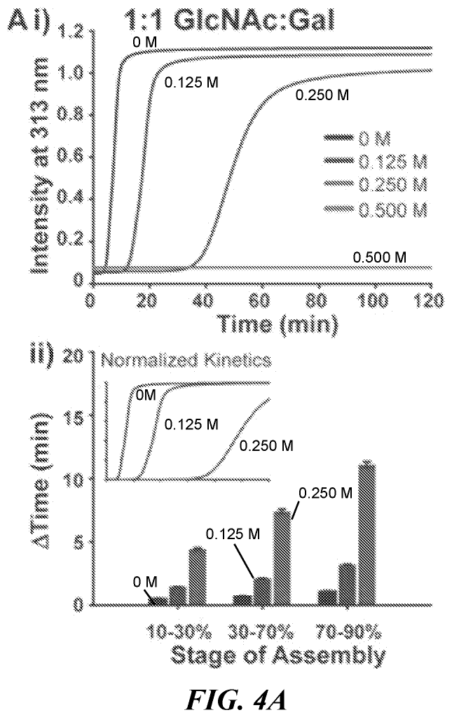

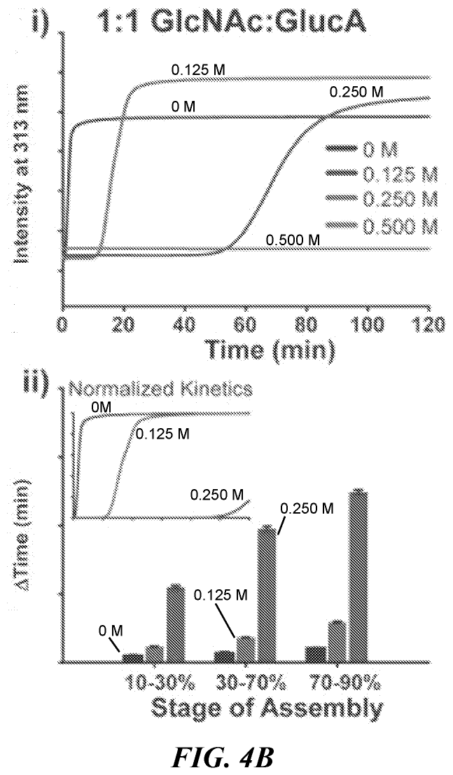

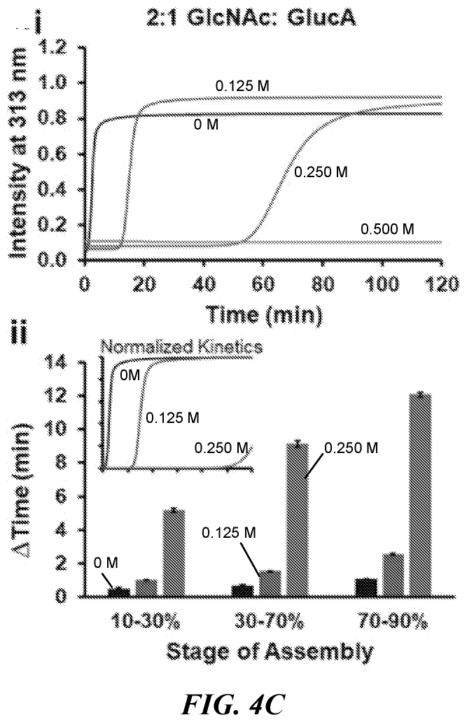

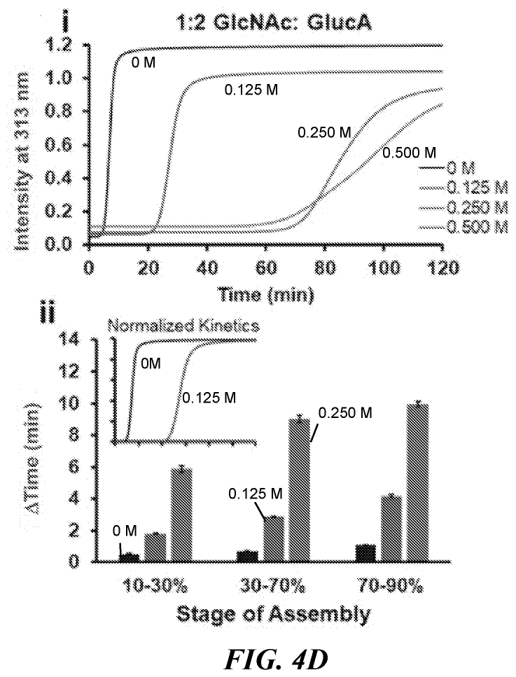

To more closely replicate the composition of GAGs present in the ECM, combinations of monosaccharides are tested using 1 mg/mL collagen, to measure assembly kinetics and complete inhibition of collagen fibrillogenesis for some concentrations of the combinations. The optical density associated with collagen fibrillogenesis is shown in response to the addition of 1:1 GlcNAc:Gal (FIG. 4A), and 1:1 GlcNAc:GlucA (FIG. 4B), 2:1 GlcNAc:GlucA (FIG. 4C), and 1:2 GlcNAc:GlucA (FIG. 4D) at varying concentrations of the saccharide combinations.

In FIG. 4A, a 1:1 ratio of GlcNAc and Gal is used to replicate the composition of KS. In FIG. 4B, a 1:1 ratio of GlcNAc and GlucA is used to replicate the composition of CS, DS, and HA, with the understanding that the saccharide is either in the disaccharide chain ora corresponding isoform. For the 2:1 ratio of GlcNAc:GlucA (FIG. 4C), the kinetics look almost identical to the 1:1 ratio (FIG. 4B), with a lag time of 63±3.6 minutes at a total saccharide concentration at 0.25 M. However, a significant change in the kinetics is observed when testing a 1:2 ratio of GlcNAc:GlucA (FIG. 4D) at 0.5 M total saccharide concentration. The saccharide solution at 0.5 M total saccharide concentration no longer completely inhibits collagen assembly as compared to the 0.5 M total saccharide concentration shown in FIG. 4A (1:1 GlcNAc:Gal). This correlates with the single saccharide experiments where GlucA is less effective at fully inhibiting collagen nucleation as shown in FIG. 2C. The kinetic data (ii, at bottom of each of FIGS. 4A-4D) are extrapolated from the turbidity curves. Insets depict the normalized turbidity for the first 60 minutes of the experiment. Normalized turbidity and kinetic are only shown for conditions where collagen assembly was not completely inhibited.

A primary difference observed in collagen kinetics with the combined saccharide solution is the increase in lag time required for assembly. At 0.25 M combined saccharide concentration for 1:1 GlcNAc:Gal (FIG. 4A), the lag phase at 30% assembly is 46±4.8 min, whereas it is respectively 16±2.5 and 15±0.6 min for either GlcNAc or Gal alone at 0.25 M. While GlucA is the least effective at delaying the rate of collagen assembly alone, it is significantly more effective at increasing the lag time in combination with GlcNAc. 1:1 GlcNAc:GlucA at 0.25 M (FIG. 4B, i, top) delays collagen lag time to 65±5.7 min, which is greater than the lag times of both the individual monosaccharide solutions and the GlcNAc:Gal combination. Additionally, an increased delay is shown in the kinetics at each stage of collagen assembly for the GlcNAc:Gal and GlcNAc:GlucA combinations (FIG. 4A and 4B, ii, bottom), as compared to the individual monosaccharides at equivalent concentrations (FIGS. 2A-2C). These data suggest a coordinated and possibly synergistic effect, wherein combinations of the monosaccharide units more effectively regulate collagen assembly in vitro.

A method for inhibiting collagen fibril formation for a type of fibrillar collagen (e.g., I, II, III, V, XI) includes providing a first solution including the individual saccharides, chains of saccharides, fragmented sections of GAGs, intact GAGs, and any of the forementioned items attached to a protein core (i.e. an full or partial proteoglycan). The solution is combined with a second solution including collagen monomers at a concentration higher than 1 μg/mL for the type of collagen. At higher collagen monomer concentrations, the resulting composition can be a gel, or can be a viscous solution resembling a gel.

Applied to healing, the high concentration threshold provided by the technology enables delivery of up to about 100 mg in 1 mL of solution, using biocompatible ingredients. Other ingredients can also be included. Live cells or other biologics can be included. It is contemplated that the technology can be utilized at a tissue healing site, at a surgical site, or for complete construction of tissue scaffolds outside a body for later implantation or surgical placement.

In an example, the resulting solution can be utilized to improve appearance of a scar, blemish, or wrinkle. An example formulation can be made by mixing the concentrated collagen solution disclosed in the form of a gel and injecting the formulation underneath the scar. The formulation can optionally contain a fibrinolysis or clot inhibitor such as aminocaproic acid. The formulation can be optionally reconstituted with a subject's plasma before being injected intradermally beneath the scar.

Dermal fillers can be utilized in the present technology, because the enhanced solubility reduces the solution viscosity and allows for delivery, for example, through smaller needles. The lack of aggregates further enables delivery through a smaller gauge needle. Many pre-existing therapies include aggregates of non-solubilized collagen. The technology disclosed herein overcomes these limitations to provide a flowable, highly concentrated collagen solution.

Other ingredients can included, such as alginate, glycerin, alcohols, phenoxyethanol, antibody-based collagen fibril inhibitors, oligonucleotides, stem cells, tissue-specific fibroblasts, (e.g., tenocytes or tendinocytes, dermal fibroblasts), epithelial cells, embryonic fibroblasts, or a patient's own cells.

In another example, the resulting solution can be contained in spheres, structures, microstructures, or nanostructures, for example including, resorbable polymethylmethacrylate (PMMA), alginate, poly(lactic-co-glycolic acid), PLGA-polyethylene, glycol-PLGA, carboxymethylcellulose, chitin, hydroxyethyl acrylate, albumin, gelatin, Pluronic F127, polyvinyl alcohol, starch, lipids, or combinations thereof.

The technology enables topical applications to dermal wounds and collagen-based tissue wounds at time of surgery (tendons, ligaments, bones, cartilage, intervertebral disks, blood vessels) or after. Cosmetic, injectable, or externally applied formulations can be formed including crosslinking agents such as glutaraldehyde, dispersants such as in phosphate-buffered physiological saline, sodium lauryl sulfate, cocamidopropyl betaine, peg-7 glyceryl cocoate, propylene glycol, peg 150 distearate, cocamide dea, and polysorbate 20.

Stabilized collagen compositions described herein can be used in conjunction with flow-induced or strain-induced assembly of collagen fibrils or other ECM structures. For example, a process such as described in US2015/0359929A1 (hereby incorporated by reference in its entirety) can be used to apply flow-induced strain, such as by a converging flow pattern in a pipette or other fluid applicator, to any stabilized collagen solution as described herein. While the saccharide, glycosaminoglycan, or other stabilizer maintains monomeric collagen in a soluble state prior to induction of mechanical strain by flow or by other means, once strain is applied to the solution, polymerization and/or fibril formation can be initiated. This method can be utilized to induce collagen fibril formation at a wound or repair site when a surgeon or other medical professional injects the stabilized collagen solution at the site, and in an orientation (e.g., controllable by flow direction) selected to provide resistance to strain and to strengthen and/or repair the tissue. The method also can be used ex vivo to print scaffolds, tissue grafts, and surrogate tissue for later use in a patient.

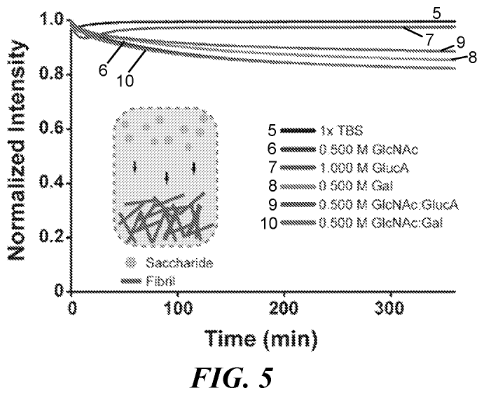

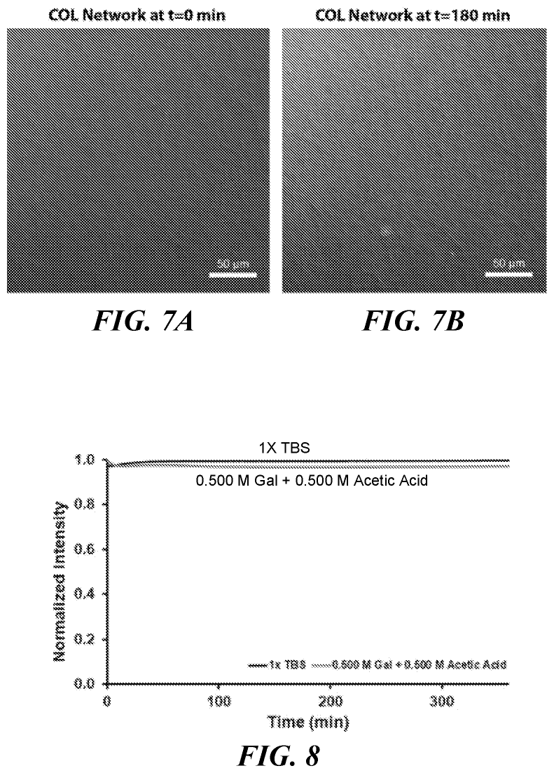

It is demonstrated that the technology can provide concentrated collagen solutions without disrupting pre-existing collagen networks. In a reverse turbidity assay, pre-established collagen networks are exposed to the critical concentrations of the saccharide solutions, and the absorbance is monitored for six hours as is shown in FIG. 5. In FIG. 5, the absorbances are normalized for comparison. After the six hour incubation period, up to an 18% decrease in the optical density of the collagen network for each saccharide condition was observed, except for GlucA alone (FIG. 5). The results from the reverse turbidity data support that collagen networks are stable in the presence of monosaccharide solutions, albeit at an altered hydration level. This finding agrees with the work of Snowden et al., where exposed collagen fibrils are exposed to CS, DS, and HA and it is determined that there is no significant change in the thermal stability of the fibrils and no effect on the integrity of the collagen fibrils (Snowden & Swann, 1980). The reverse turbidity assays presented in FIG. 5 are repeated on an inverted microscope. FIG. 7A shows an untreated control collagen network. FIG. 7B shows the network after exposure to Gal at critical concentration to suppress collagen assembly, and little to no change is observed. The images in FIG. 7A and FIG. 7B are representative of all observed conditions.

Although fibril hydration is not directly measured in the reverse turbidity assay, the findings correlate with a phenomenon known as optical clearing. Optical clearing in collagen is caused by fibril dehydration, thus decreasing the density of a fibrillar network (Wen, X., et al., 2010; Yeh, A. T., et al., 2003; Hovhannisyan, V., et al., 2013; Hirshburg, J., et al., 2007). Glycerol and sugars, such as sucrose and fructose, have been shown to cause this effect on different collagenous tissues (Wen, X., et al., 2010; Hovhannisyan, V., et al., 2013; Hirshburg, J., et al., 2007). Furthermore, the optical density can be restored after rinsing the tissue with buffer, which suggests a non-destructive mechanism (Yeh, A. T., et al., 2003). Taken together, this indicates that the decrease in collagen's optical density in response to the addition of a monosaccharide solution is similarly caused by fibril dehydration induced by the hygroscopic nature of the monosaccharides.

In the reverse turbidity plots shown in FIG. 5, the plateau is interpreted, with the absence of recognizable fibril dissociation (e.g., under microscope) to suggest a change in the hydration level of the fibrils in all cases except for the GlucA condition, which it is suspected relates to the negatively charged functional group (i.e., the carboxylic acid of GlucA, which was deprotonated due to a 3.2 pKa value). To support this hypothesis, the experiment with a 1:1 combination of Gal and acetic acid to replicate the negatively charged environment was repeated in the reverse turbidity assay presented in FIG. 8. The results in FIG. 8 show only a slight (3%) decrease in optical density compared to the control, resembling the profile of GlucA, further supporting an important role for charge on pre-existing collagen fibrils.

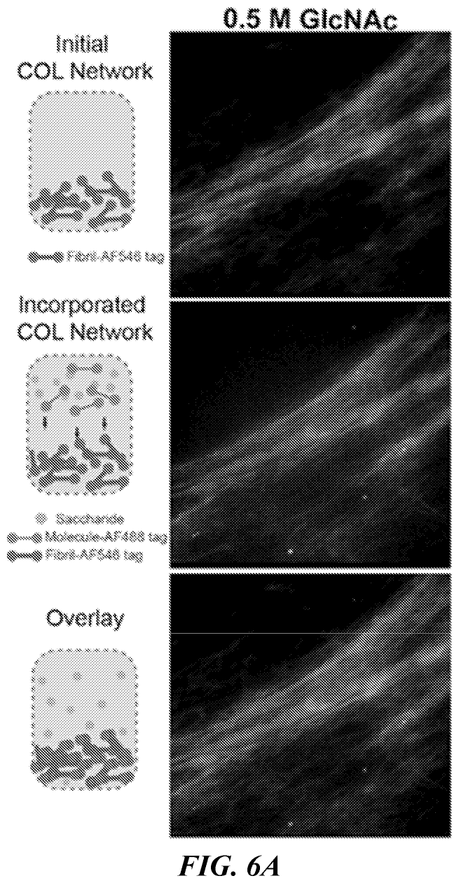

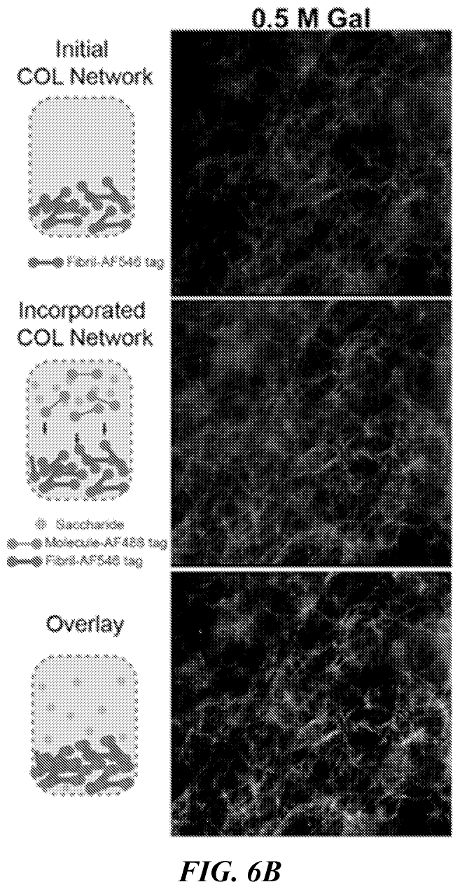

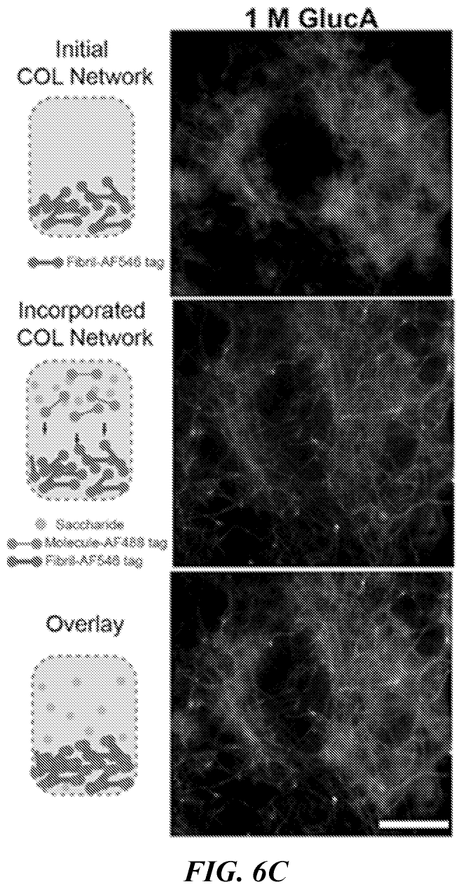

While the technology provides high concentrations of collagen in solution with inhibition of spontaneous collagen nucleation, it is demonstrated herein that collagen nucleation can be inhibited without disrupting incorporation and fibril growth. As is shown by the dual fluorescence study presented in FIGS. 6A-6C, collagen molecules, when mixed with critical concentrations of saccharides, maintained functionality and successfully incorporated into a pre-existing matrix. There are two reasons why it is suspected this is achievable. First, as demonstrated by Comper and Veis, the activation energy required for fibrillogenesis is reduced when pre-formed nucleators are present (Comper & Veis, 1977). Secondly, as explored by Evans and Drouven, the addition of modifying solutes can affect the activation energies associated with nucleation and growth independently, whereby growth may become favorable despite the nucleation inhibition (Evans & Drouven, 1983). Collagen incorporation in the presence of the inhibitory saccharides supports the possibility that GAGs provide a means for rapid repair of fibrillar micro-damage in vivo and will require further studies with full GAGs to better characterize. Furthermore, it raises the question as to whether there is coordinated guidance with the PGs found decorating the fibril surfaces. Given their capacity to regulate fibril diameter, it is questioned if healthy tissue has minimal incorporation of collagen due to PG shielding, whereas an injury site with displaced PGs may preferentially focus the incorporation toward repair.

The saccharides which make up the GAG chains present in the ECM substantially alter collagen assembly kinetics, solubility, and incorporation, as compared to a pure collagen solution in vitro. Spectrophotometry is utilized to determine the critical concentration of monosaccharide needed to inhibit collagen nucleation and to explore the effects that different combinations of monosaccharides had on assembly kinetics. It is shown that the monosaccharides do not destabilize or degrade the collagen fibrils after assembly, and fluorescent imaging reveals that collagen growth and incorporation into a pre-existing network is done in nucleation-inhibiting conditions. Notably, without the need to deviate from a physiologically relevant environment, it is demonstrated herein the capability to raise the solubility of collagen by about 3-5 orders of magnitude, while preserving its ability to integrate into pre-existing collagen networks. The present technology provides the use of GAGs to regulate a reserve of soluble collagen in the interstitial fluid of the ECM for immediate availability. Furthermore, the substantial concentration increase for stable, monomeric collagen in vitro supports a broad range of applications such as 3-D tissue printing, cell and tissue cultivation, and therapeutic technologies.

### EXAMPLES

### Example 1

### Influence of Monosaccharides on Collagen Polymerization

Stock solutions of N-acetyl-D-glucosamine (MP Biomedicals, 0210006880), D-glucuronic acid sodium salt monohydrate (Acrōs Organics, 20457-1000), and D(+)-galactose (Acrōs Organics, 15061-1000) were made near maximum solubility at 1, 2, and 2 M, respectively. Each powder was dissolved in deionized water at 37° C. with constant stirring, adjusted to pH 5.0 with the addition of hydrochloric acid, and brought up to the proper volume using a volumetric flask. An acidic pH was selected to minimize immediate collagen polymerization prior to the mixing of the reagents. A 5 M stock solution of acetic acid was made by dilution of glacial acetic acid (Fisher Chemical, A38S-500) in a volumetric flask and adjusted to pH 6.0 with sodium hydroxide.

In the experiments below, acetic acid-extracted bovine type I collagen (Advanced Biomatrix, 5026-1 KIT) was mixed with monosaccharide solutions, and/or acetic acid, such that the final collagen concentration was either 0.5 or 1 mg/mL and final saccharide concentration ranged from 0-1 M. Concentrated Tris-buffered saline (10×TBS) at 0.25 M trizma base (Sigma Aldrich, T4661) and 1.3 M sodium chloride

(Fisher Chemical, S271-500), was used as 10% of the solution volume to maintain a physiological pH and ionic strength, and 0.25 M sodium hydroxide was used for a final pH adjustment to 7.3. Any remaining volume in the solution recipes was fulfilled via the addition of deionized water.

To decouple the complexities associated with variable surface charge, size, and branching of native GAGs, the experiments showed how the monosaccharides (GlcNAc, Gal, and GlucA) found in GAGs affected collagen assembly kinetics in vitro. Experiments included mixing collagen with a concentrated monosaccharide solution, neutralizing it, and measuring turbidity spectrophotometrically (313 nm) over the course of two hours. In the absence of monosaccharides, 1 mg/mL collagen fully polymerized within minutes, as expected (Williams, B. R., et al., 1978). However, in the presence of concentrated monosaccharides, the assembly kinetics were significantly delayed and completely inhibited for some concentrations. Using 1 mg/mL collagen, as shown in FIG. 2A and FIG. 2B, 0.125 M GlcNAc and 0.125 M Gal each produced a delay in the lag phase and growth phase and an increase in the maximum absorbance (ODₘₐₓ) of collagen. The increased ODₘₐₓ caused by the monosaccharides cannot be fully attributed to the contribution of the monosaccharides in solution (Table 2) (Hayashi & Nagai, 1972; Wood & Keech, 1960). The same initial absorbance for GlcNAc and Gal, as compared to the collagen-only condition, suggest a modified fibrillar architecture. In FIG. 2A and FIG. 2B, kinetics were further delayed with increasing concentration of GlcNAc and Gal until collagen assembly was completely inhibited at a critical concentration of 0.5 M.

TABLE 2
Baseline Absorbance Values for the Monosaccharides

| [Saccharide] | GlcNAc | GlucA | Gal |
|---|---|---|---|
| 0.125M | 0.061 ± 0.001 | — | 0.041 ± 0.001 |
| 0.25M | 0.074 ± 0.001 | 0.063 ± 0.000 | 0.043 ± 0.001 |
| 0.5M | 0.105 ± 0.001 | 0.082 ± 0.001 | 0.044 ± 0.001 |
| 1M | — | 0.120 ± 0.000 | — |

In Table 2, The absorbance of each monosaccharide for each experimental concentration was measured at 313 nm at 37° C. No collagen was present in these samples. The average and standard deviation for n=3 of each solution is displayed above.

To study the assembly of collagen, the kinetics of collagen assembly in the presence of monosaccharides were monitored using a plate-reader spectrophotometer (Molecular Devices, SpectraMax M5). Working in triplicate, 100 μL of the mixed collagen and saccharide solution was pipetted into a 96-well plate and layered with 100 μL of silicone oil to prevent evaporation. The samples were placed in the preheated 37° C. spectrophotometer, and optical density was measured at 313 nm every 30 seconds for 2 hours. Each test condition was repeated three times for a total of n=9 data sets.

The analyze the assembly kinetics, turbidity assays were analyzed and normalized between 0-1 using Equation 1, where OD represents the optical density.

$$Normalized Absorbance = \frac{O {D}_{313 nm} - O {D}_{min}}{O {D}_{max} - O {D}_{min}} \qquad Equation 1$$

To analyze the kinetics, 0% assembly was defined as the absorbance value when the minimum absorbance was recorded, and 100% corresponded to the maximum value. 10, 30, 70 and 90% absorbance and corresponding timepoints were calculated using linear interpolation between the two bounding data points. Error bars were calculated as standard error of the mean. Rate of assembly was determined by calculating the slope between 30% and 70% assembly for each data set. The average rates and standard deviations are reported in Table 3.

TABLE 3
Rate of Collagen Assembly in the Presence of Monosaccharides
Rates of Collagen Assembly (K_{A})

| | Monosaccharides | | | | |
|---|---|---|---|---|---|
| Controls | [Saccharide] | GlcNAc | Gal | [Saccharide] | GlucA |
| Collagen | | | | | |
| 1 mg/mL | | | | | |
| 56.42 ± 11.67% | 0.125M | 17.91 ± 1.52% | 28.71 ± 1.54% | 0.25M | 13.83 ± 1.10% |
| | 0.25M | 8.27 ± 0.54% | 15.96 ± 1.39% | 0.5M | 40.64 ± 2.59% |
| Collagen | | | | | |
| 0.5 mg/mL | | | | | |
| 43.62 ± 5.22% | 0.125M | 13.32 ± 0.63% | 14.28 ± 1.48% | 0.25M | 6.84 ± 0.47% |
| | 0.25M | 6.77 ± 0.31% | 7.22 ± 0.89% | 0.5M | 10.69 ± 0.53% |

In Table 3, the average rate and standard deviation of collagen polymerization from 30% to 70% was calculated from the turbidimetric data shown in FIGS. 2A-2F. The results are n=9 and reported as % per second. Gal=galactose; GlcNAc=N-acetyl-glucosamine; GlucA=glucuronic acid.

In FIGS. 2A-2C, experiments were conducted with 1 mg/mL collagen. In FIGS. 2D-2F, experiments were repeated with 0.5 mg/mL collagen. In FIGS. 2A-2F, optical density (i, top of each Figure) was measured in the presence of GlcNAc (FIG. 2A, FIG. 2D), Gal (FIG. 2B, FIG. 2E), and GlucA (FIG. 2C, FIG. 2F). The kinetic data (ii, bottom of each Figure) were extrapolated from the turbidity curves for GlcNAc, Gal, and GlucA. Insets represent the normalized turbidity over 60 minutes. Normalized turbidity and kinetic data was not represented for conditions where collagen assembly was completely inhibited. The data represents the average of 9 runs, and the kinetic data error bars represent the standard error of the mean.

Although GlcNAc and Gal similarly affected the assembly of collagen, GlucA exhibited one key difference: it required twice the molar concentration to inhibit collagen assembly (FIG. 2C, top, i). Additionally, GlucA had a non-linear effect on delaying assembly kinetics (FIG. 2C, bottom, ii). When GlucA was added to the collagen solution for the higher concentration recipes (e.g., 0.5 and 1 M), there was a small amount of spontaneous collagen assembly as an artifact of the mixing process, as evidenced by the increased flatline absorbance for 1 M GlucA (FIG. 2C, top, i). The presence of spontaneous collagen assembly with GlucA is potentially due to an interaction between the positively charged collagen and the negatively charged carboxylate group of GlucA (Zemann, A., et al., 1997; Wang, X., et al., 2012; Uquillas & Akkus, 2012). This electrostatic interaction may also account for the shorter lag time for 0.5 M compared to 0.25 M GlucA, as well as the lessened effect on delaying the kinetics. Although these aggregates may be seeding collagen nucleation, the rate of kinetic assembly was still delayed compared to the collagen-only condition, and the steady-state absorbance was still increased.

When experiments were repeated with 0.5 mg/mL collagen (FIGS. 2D-2F), the concentration of monosaccharide required to inhibit collagen assembly was constant for each saccharide. Interestingly, the effect that these monosaccharides have on inhibiting collagen assembly was independent of collagen concentration. The increased lag times, delayed assembly kinetics, and heightened steady-state absorbance profiles were similarly observed for each condition at both 0.5 and 1 mg/mL collagen. This suggests an indirect mechanism for modulating collagen assembly that requires a critical concentration of saccharide rather than a molecular ratio to collagen for complete inhibition.

### Example 2

### Influence of Negatively Charged Species on Collagen Polymerization

To further explore the effects of electrostatic interactions on collagen assembly, an investigation of whether the addition of an auxiliary negative charge (i.e., acetic acid) to the Gal condition could replicate the influence that GlucA has on collagen polymerization was designed. To test this, collagen assembly (1 mg/mL) was monitored on the plate-reader with the two additional conditions of 0.5 M acetic acid alone and with 0.5 M Gal/0.5 M acetic acid (FIG. 3A). FIG. 3A shows the results of the turbidity assay to observe the influence of acetic acid on collagen assembly directly and in conjunction with the monosaccharide Gal. Acetic acid provided the negative charge present on GlucA but absent from Gal. As shown in FIG. 3A, acetic acid alone had little impact on the early kinetics but significantly reduced the steady-state absorbance as compared to the collagen-only condition. Given the compromised capacity of collagen to assemble in the presence of acetic acid, it was anticipated that the addition of acetic acid and Gal would continue to fully inhibit collagen polymerization. However, even though 0.5 M Gal had completely inhibited collagen assembly, as is reshown at the bottom of FIG. 3A, the combination of Gal and acetic acid partially restored collagen's ability to spontaneously polymerize. Kinetic data for select conditions was compared and is shown in FIG. 3B. The inset of FIG. 3B shows the normalized turbidity data over 60 minutes. Error bars represent the standard error of the mean (n=9). While the kinetics of 0.5 M Gal with 0.5 M acetic acid do not fully match those of 0.5 M GlucA (FIG. 3B), this may be attributed to the negative charge of the acetic acid not being directly attached to the Gal, as is the case with GlucA. Given that acetic acid was an assembly inhibitor for one condition but a catalyst for the other, the exact role of electrostatic interactions on collagen nucleation and growth remains for further studies. Still, it is suspected that charge plays an important role of charge in regulating fibril formation.

### Example 3

### Influence of Combined Monosaccharides on Collagen Polymerization

Combinations of monosaccharides were tested to more closely replicate the composition of GAGs present in the ECM. The optical density associated with collagen fibrillogenesis was measured in response to the addition of 1:1 GlcNAc:Gal (FIG. 4A), and 1:1 GlcNAc:GlucA (FIG. 4B), at varying concentrations.

A 1:1 ratio of GlcNAc and GlucA was used to replicate the composition of CS, DS, and HA, with the understanding that the saccharide used was either in the disaccharide chain or a corresponding isoform. Similarly, a 1:1 ratio of GlcNAc and Gal was used to replicate the composition of KS. In these experiments, reported concentrations for each 1:1 ratio, ranging from 0-0.5 M, represented the combined molarity of the saccharides, whereby each monosaccharide contributes to half the combined concentration.

The effect of combined saccharides, by altering the ratio of GlcNAc and GlucA in solution with collagen was further explored (FIG. 4C, FIG. 4D). The optical density associated with collagen fibrillogenesis was measured in response to the addition of 2:1 GlcNAc:GlucA (FIG. 4C), and 1:2 GlcNAc:GlucA (FIG. 4D), at varying concentrations. For a 2:1 ratio of GlcNAc:GlucA with the total saccharide concentration at 0.25 M, the kinetics looked almost identical to the 1:1 ratio, with a lag time of 63±3.6 min. However, a significant change in the kinetics was observed when testing the 1:2 ratio of GlcNAc:GlucA at 0.5 M total saccharide concentration. The saccharide solution no longer completely inhibited collagen assembly. This correlated with the single saccharide experiments where GlucA was less effective at fully inhibiting collagen nucleation.

The kinetic data (ii, at bottom of each figure) were extrapolated from the turbidity curves. Insets depict the normalized turbidity for the first 60 minutes of the experiment. Normalized turbidity and kinetic data were only for conditions where collagen assembly was not completely inhibited. The data represents the average of 9 runs, and the kinetic data error bars represent the standard error of the mean. Table 4 presents a summary of the rates of collagen assembly.

TABLE 4
Rate of Collagen Assembly in the Presence of Saccharide Ratios
Rates of Collagen Assembly (K_{A})

| Controls | Saccharide Ratios | | | | |
|---|---|---|---|---|---|
| Collagen | | 1:1 | 1:1 | 2:1 | 1:2 |
| 1 mg/mL | [Saccharide] | GlcNAc:GlucA | GlcNAc:Gal | GlcNAc:GlucA | GlcNAc:GlucA |
| 56.42 ± 11.67% | 0.125M | 20.71 ± 1.27% | 18.31 ± 0.68% | 24.97 ± 1.93% | 13.41 ± 0.76% |
| | 0.25M | 4.05 ± 0.22% | 5.34 ± 0.38% | 4.35 ± 0.30% | 4.41 ± 0.28% |

In Table 4, the average rate and standard deviation of collagen polymerization from 30% to 70% was calculated from the turbidimetric data shown in FIGS. 4A-4D. The results are n=9 and reported as % per second. Gal=galactose; GlcNAc=N-acetyl-glucosamine; GlucA=glucuronic acid.

A primary difference observed in collagen kinetics with the combined saccharide solution was the increase in lag time required for assembly. At 0.25 M combined saccharide concentration for 1:1 GlcNAc:Gal (FIG. 4A), the lag phase at 30% assembly was 46±4.8 min, whereas it was respectively 16±2.5 and 15±0.6 min for either GlcNAc or Gal alone at 0.25 M. While GlucA was the least effective at delaying the rate of collagen assembly alone, it was significantly more effective at increasing the lag time in combination with GlcNAc. 1:1 GlcNAc:GlucA at 0.25 M (FIG. 4B, i, top) delayed collagen lag time to 65±5.7 min, which was greater than the lag times of both the individual monosaccharide solutions and the GlcNAc:Gal combination. Additionally, an increased delay is observed in the kinetics at each stage of collagen assembly for the GlcNAc:Gal and GlcNAc:GlucA combinations (FIG. 4A and 4B, ii, bottom), as compared to the individual monosaccharides at equivalent concentrations (FIGS. 2A-2C). These data suggest a coordinated and possibly synergistic effect, wherein combinations of the monosaccharide units more effectively regulate collagen assembly in vitro.

### Example 4

### Effects of Saccharides on Pre-Existing Collagen Matrix

While previous experiments revealed that monosaccharides can completely inhibit collagen assembly, it was unknown how these critical concentrations would affect pre-existing collagen networks. This was studied using a reverse turbidity assay, where pre-established collagen networks were exposed to the critical concentrations of saccharide solutions, and the absorbance was monitored for six hours. After this incubation period, up to an 18% decrease in the optical density of the collagen network for each saccharide condition was observed, except for GlucA alone (FIG. 5). In FIG. 5, the change in absorbance of a pre-established collagen network was measured at 313 nm over six hours at 37° C. in response to the addition of various saccharide solutions. The data for each condition (n=9) has been normalized to a 0-1 scale for comparison.

The reverse turbidity assays were conducted using the following procedure. 1 mg/mL collagen networks were polymerized in a closed 96-well plate for 30 minutes at 37° C. Following complete polymerization, pH 7.3 solutions of 1× TBS (control, top of FIG. 5), the three individual monosaccharides, and the two 1:1 ratios were each carefully pipetted on top of the collagen networks in triplicate. A layer of silicone oil was then pipetted on top of each well to prevent evaporation while scanning in the plate-reader. The same process was repeated later with acetic acid and a mixture of acetic acid and Gal. Optical density was measured at 313 nm over 6 hours to assess any change in turbidity that would be indicative of collagen fibril dissociation. Data were normalized between 0-1 and plotted using equation (2).

$$Normalized Absorbance = \frac{O {D}_{313 nm}}{O {D}_{max}} \qquad Equation 2$$

To further investigate fibril dissociation, the experiments were repeated on a microscope to monitor physical changes to the networks in each condition; however, no visible change in the fibrillar network was identified. An example is shown in FIG. 7A and FIG. 7B, which show representative microscope images of collagen network before and after exposure to Gal. The reverse turbidity experiments were performed with an inverted microscope to observe changes in the collagen network at 37° C. for 180 minutes. The images illustrated minimal to no change in the network after exposure to Gal. The images in FIG. 7A and FIG. 7B are representative of all conditions.

As such, the plateau was interpreted in the reverse turbidity plots (FIG. 5) and the absence of recognizable fibril dissociation to suggest a change in the hydration level of the fibrils in all cases except for the GlucA condition, which it was suspected relates to the negatively charged functional group. To support this hypothesis, the experiment with a 1:1 combination of Gal and acetic acid to replicate the negatively charged environment was repeated (FIG. 8). The results in FIG. 8 showed only a slight (3%) decrease in optical density compared to the control, resembling the profile of GlucA, further supporting an important role for charge on pre-existing collagen fibrils.

### Example 5

### Collagen Incorporation into a Pre-Existing Network in the Presence of Monosaccharides

After observing that saccharides inhibited assembly without significantly disrupting pre-existing collagen networks, experiments were designed to study if the critical saccharide concentrations would allow the soluble collagen to incorporate into a pre-existing matrix, despite the observed nucleation inhibition.

Collagen was fluorescently labeled by the following procedure. Collagen was amine-labeled with either Alexa Fluor 546 NHS Ester (Thermo Fisher Scientific, A20002) or Alexa Fluor 488 TFP (Thermo Fisher Scientific, A30005) according to the protocol of Paten et al. (Paten, J. A., et al., 2019). Collagen was labeled with A488 solution in a 5:1 molar ratio of A488:collagen and then purified using a 10,000 MWCO Slide-A-Lyzer dialysis cassette (Thermo Fisher Scientific, 66380). The same process was performed with A546 in a 5:1 molar ratio of A546:collagen. Prior to use, the collagen concentration was determined by a modified Lowry assay (Bio-Rad, DC-Protein Assay), and the labeling efficiency was determined according to previous reports (Paten, J. A., et al., 2019). Calculations showed a final molar ratio of 1.0:1 A488:collagen and 1.5:1 A546:collagen.

To test if the critical saccharide concentrations would allow the soluble collagen to incorporate into a pre-existing matrix, despite the observed nucleation inhibition, first a collagen network was polymerized labeled with a A546 (red) at 37° C. using the example procedure described above. Then a mixture of A488-labeled collagen (green) with a nucleation-inhibiting concentration of either GlcNAc, Gal, or GlucA was added to the pre-existing A546-labeled collagen network. In all cases, distinct fibrillar networks of A488-collagen after a 1 hour incubation and exhaustive rinsing to reduce non-specific binding was observed.

The ability for soluble collagen to incorporate into a pre-established collagen network in the presence of monosaccharides was investigated with fluorescent imaging using an inverted microscope (Nikon, ECLIPSE Ti2) with a 0.45 numerical aperture 20× objective (Nikon, MRH08230) and a CMOS camera (Andor, Zyla 4.2). All the experiments were performed in a Delta-T dish (Bioptechs, 04200417C) that was pretreated with 3% bovine serum albumin for one hour to prevent any non-specific binding on the glass. The dish was rinsed out with 1× TBS, and then 0.75 mL of neutralized 0.025 mg/mL, A546-labeled collagen solution was added to the dish. The concentration was chosen to generate a sparse network for clarity during fluorescent imaging. The A546-collagen network was heated to 37° C. and given 45 min to assemble.

A488-labeled collagen was mixed with one of the three saccharides, such that when added to the 0.75 mL solution volume in the Delta-T dish, the saccharide concentration matched the critical concentration required to stop spontaneous assembly. The soluble collagen final concentration was 0.025 mg/mL. After a 1 hour incubation at 37° C., the network was rinsed 20 times by removing 0.75 mL of solution from the dish and adding 0.75 mL of saccharide-only solution. This process reduced the background noise and washed off any loosely bound collagen. Images were then captured by isolating the A488 and A546 fluorophores, from which an overlay image was generated. This process was repeated with each saccharide (GlcNAc, Gal, and GlucA) to evaluate collagen's ability to incorporate into a pre-existing matrix.

The Imaging and Colocalization Analysis of the Dual-Fluorescence collagen Network was conducted using the following procedure. For imaging the A546-labeled collagen network, the excitation signal (CoolLED, PE-300-W-L-SB-40) was passed through a 530±15 nm filter, and the emission signal was detected after passing through a 575±20 nm filter (Chroma Technology, 49014). For imaging the A488-labeled collagen, the excitation signal passed through a 470±20 nm filter, and the emission signal was captured after passing through a 525±25 nm filter (Chroma Technology, 49002). The networks were imaged at ten different locations with a 20× objective lens, where the pre-established network was sparse enough to image both the pre-existing and incorporated network with minimal interference from any out of plane network. A Pearson's Correlation Coefficient (PCC) score was generated for each image by the Nikon Eclipse software to measure the degree of colocalization and the average PCC score and standard deviation were calculated. The PCC score ranged from −1 to 1, where 1 was an exact positive correlation, 0 was no correlation, and −1 was a perfect negative correlation (K. W., D., et al., 2011). Out of focus regions were cropped from the images to improve scoring accuracy.

The fluorophore images of FIGS. 6A-6C show the incorporation of collagen into a pre-existing network in the presence of the critical monosaccharide concentrations. The top rows of FIGS. 6A-6C display images of the pre-polymerized, fibrous A546-labeled collagen (COL) network. The middle rows captures the same location but identifies the A488-labeled collagen that was added as mixture with the monosaccharides. The bottom rows shows the overlay product of the two isolated fluorophore images. The experiment was carried out in the presence of either 0.5 M GlcNAc (FIG. 6A), 0.5 M Gal (FIG. 6B), or 1 M GlucA (FIG. 6C). These results indicated that collagen, in the presence of monosaccharides, was still able to incorporate into fibrils but required a pre-existing network to build on. The incorporation efficiency of the new fibrils with the pre-established network was analyzed using the Pearson's correlation coefficients (PCC). PCC values of 0.66±0.10 for GlcNAc, 0.53±0.11 for Gal, and 0.50±0.11 for GlucA (n=10 images) were observed. The moderate PCC scores indicated that while A488-collagen did integrate into the pre-existing matrix, there was selective incorporation rather than layering on every fibril. This was expected, in that not every fibrillar surface was expected to be receptive to further growth. Nonetheless, the combined results demonstrated that monosaccharides were capable of dual functionality, whereby collagen nucleation could be prevented, yet functional incorporation into pre-existing structures could be preserved.

### REFERENCES

Iozzo, R. V.; Schaefer, L., Proteoglycan form and function: A comprehensive nomenclature of proteoglycans. Matrix Biol 2015, 42, 11-55.
Schaefer, L.; lozzo, R. V., Biological functions of the small leucine-rich proteoglycans: from genetics to signal transduction. J Biol Chem 2008, 283 (31), 21305-9.
Silbert, J. E.; Sugumaran, G., Biosynthesis of chondroitin/dermatan sulfate. IUBMB Life 2002, 54 (4), 177-86.
Sugahara, K.; Kitagawa, H., Heparin and heparan sulfate biosynthesis. IUBMB Life 2002, 54 (4), 163-75.
Brown, S.; Melrose, J.; Caterson, B.; Roughley, P.; Eisenstein, S. M.; Roberts, S., A comparative evaluation of the small leucine-rich proteoglycans of pathological human intervertebral discs. Eur Spine J 2012, 21 Suppl 2, S154-9.
Hassell, J. R.; Kimura, J. H.; Hascall, V. C., Proteoglycan core protein families. Annu Rev Biochem 1986, 55, 539-67.
Mende, M.; Bednarek, C.; Wawryszyn, M.; Sauter, P.; Biskup, M. B.; Schepers, U.; Brase, S., Chemical Synthesis of Glycosaminoglycans. Chem Rev 2016, 116 (14), 8193-255.
Prydz, K.; Dalen, K. T., Synthesis and sorting of proteoglycans. J Cell Sci 2000, 113 Pt 2, 193-205.
Caniggia, I.; Tanswell, K.; Post, M., Temporal and spatial differences in glycosaminoglycan synthesis by fetal lung fibroblasts. Exp Cell Res 1992, 202 (2), 252-8.
Li, L.; Ly, M.; Linhardt, R. J., Proteoglycan sequence. Mol Biosyst 2012, 8 (6), 1613-25.
Chen, S.; Birk, D. E., The regulatory roles of small leucine-rich proteoglycans in extracellular matrix assembly. FEBS J 2013, 280 (10), 2120-37.
Scott, J. E., Proteoglycan-fibrillar collagen interactions. Biochem J 1988, 252 (2), 313-23.
Miyagawa, A.; Kobayashi, M.; Fujita, Y.; Hamdy, O.; Hirano, K.; Nakamura, M.; Miyake, Y., Surface ultrastructure of collagen fibrils and their association with proteoglycans in human cornea and sclera by atomic force microscopy and energy-filtering transmission electron microscopy. Cornea 2001, 20 (6), 651-6.
Pringle, G. A.; Dodd, C. M., Immunoelectron microscopic localization of the core protein of decorin near the d and e bands of tendon collagen fibrils by use of monoclonal antibodies. J Histochem Cytochem 1990, 38 (10), 1405-11.
Zhang, G.; Chen, S.; Goldoni, S.; Calder, B. W.; Simpson, H. C.; Owens, R. T.; McQuillan, D. J.; Young, M. F.; lozzo, R. V.; Birk, D. E., Genetic evidence for the coordinated regulation of collagen fibrillogenesis in the cornea by decorin and biglycan. J Biol Chem 2009, 284 (13), 8888-97.
Melrose, J.; Ghosh, P.; Taylor, T. K., A comparative analysis of the differential spatial and temporal distributions of the large (aggrecan, versican) and small (decorin, biglycan, fibromodulin) proteoglycans of the intervertebral disc. JAnat 2001, 198 (Pt 1), 3-15.
Dunkman, A. A.; Buckley, M. R.; Mienaltowski, M. J.; Adams, S. M.; Thomas, S. J.; Satchell, L.; Kumar, A.; Pathmanathan, L.; Beason, D. P.; lozzo, R. V.; Birk, D. E.; Soslowsky, L. J., The tendon injury response is influenced by decorin and biglycan. Ann Biomed Eng 2014, 42 (3), 619-30.
Chakravarti, S., Functions of lumican and fibromodulin: lessons from knockout mice. Glycoconj J 2002, 19 (4-5), 287-93.
Vogel, K. G.; Paulsson, M.; Heinegard, D., Specific inhibition of type I and type II collagen fibrillogenesis by the small proteoglycan of tendon. Biochem J 1984, 223 (3), 587-97.
Chakravarti, S.; Zhang, G.; Chervoneva, I.; Roberts, L.; Birk, D. E., Collagen fibril assembly during postnatal development and dysfunctional regulation in the lumican-deficient murine cornea. Dev Dyn 2006, 235 (9), 2493-506.
Liu, C. Y.; Birk, D. E.; Hassell, J. R.; Kane, B.; Kao, W. W., Keratocan-deficient mice display alterations in corneal structure. J Biol Chem 2003, 278 (24), 21672-7.
Chakravarti, S.; Petroll, W. M.; Hassell, J. R.; Jester, J. V.; Lass, J. H.; Paul, J.; Birk, D. E., Corneal opacity in lumican-null mice: defects in collagen fibril structure and packing in the posterior stroma. Invest Ophthalmol Vis Sci 2000, 41 (11), 3365-73.
Chakravarti, S.; Magnuson, T.; Lass, J. H.; Jepsen, K. J.; LaMantia, C.; Carroll, H., Lumican Regulates Collagen Fibril Assembly: Skin Fragility and Corneal Opacity in the Absence of Lumican. The Journal of Cell Biology 1998, 141 (5), 1277-1286.
Ameye, L.; Aria, D.; Jepsen, K.; Oldberg, A.; Xu, T.; Young, M. F., Abnormal collagen fibrils in tendons of biglycan/fibromodulin-deficient mice lead to gait impairment, ectopic ossification, and osteoarthritis. FASEB J 2002, 16 (7), 673-80.
Fust, A.; LeBellego, F.; lozzo, R. V.; Roughley, P. J.; Ludwig, M. S., Alterations in lung mechanics in decorin-deficient mice. Am J Physiol Lung Cell Mol Physiol 2005, 288 (1), L159-66.
Zhang, G.; Ezura, Y.; Chervoneva, I.; Robinson, P. S., Beason, D. P.; Carine, E. T.; Soslowsky, L. J.; lozzo, R. V.; Birk, D. E., Decorin regulates assembly of collagen fibrils and acquisition of biomechanical properties during tendon development. J Cell Biochem 2006, 98 (6), 1436-49.
Corsi, A.; Xu, T.; Chen, X. D.; Boyde, A.; Liang, J.; Mankani, M.; Sommer, B.; lozzo, R. V.; Eichstetter, I.; Robey, P. G.; Bianco, P.; Young, M. F., Phenotypic effects of biglycan deficiency are linked to collagen fibril abnormalities, are synergized by decorin deficiency, and mimic Ehlers-Danlos-like changes in bone and other connective tissues. J Bone Miner Res 2002, 17 (7), 1180-9.
Kalamajski, S.; Bihan, D.; Bonna, A.; Rubin, K.; Famdale, R. W., Fibromodulin Interacts with Collagen Cross-linking Sites and Activates Lysyl Oxidase. J Biol Chem 2016, 291 (15), 7951-60.
Maruhashi, T.; Kii, I.; Mitsuru, S.; Kudo, A., Interaction between Periostin and BMP-1 Promotes Proteolytic Activation of Lysyl Oxidase. J Biol Chem 2010, 285 (17), 13294-13303.
Geng, Y.; McQuillan, D.; Roughley, P. J., SLRP interaction can protect collagen fibrils from cleavage by collagenases. Matrix Biol 2006, 25, 484-491.
Pietraszek, K.; Chatron-Colliet, A.; Brezillon, S.; Perreau, C.; Jakubiak-Augustyn, A.; Krotkiewski, H.; Maquart, F. X.; Wegrowski, Y., Lumican: a new inhibitor of matrix metalloproteinase-14 activity. FEBS Lett 2014, 588 (23), 4319-24.
Kalamajski, S.; Oldberg, A., The role of small leucine-rich proteoglycans in collagen fibrillogenesis. Matrix Biol 2010, 29 (4), 248-53.
Han, E. H.; Chen, S. S.; Klisch, S. M.; Sah, R. L., Contribution of proteoglycan osmotic swelling pressure to the compressive properties of articular cartilage. Biophys J 2011, 101 (4), 916-24.
Ruiz Hernandez, S. E.; Streeter, I.; de Leeuw, N. H., The effect of water on the binding of glycosaminoglycan saccharides to hydroxyapatite surfaces: a molecular dynamics study. Phys Chem Chem Phys 2015, 17 (34), 22377-88.
Harvey, J. M.; Symons, M. C. R., The Hydration of Monosaccharides—an NMR Study. J Solution Chem 1978, 7 (8).
Petukhov, M.; Rychkov, G.; Firsov, L.; Serrano, L., H-bonding in protein hydration revisited. Protein Sci 2004, 13 (8), 2120-9.
Huggins, D. J., Quantifying the entropy of binding for water molecules in protein cavities by computing correlations. Biophys J 2015, 108 (4), 928-936.
Li, Z.; Lazaridis, T., Thermodynamic contributions of the ordered water molecule in HIV-1 protease. J Am Chem Soc 2003, 125 (22), 6636-7.
Cooper, A., Thermodynamic studies of the assembly in vitro of native collagen fibrils. Biochem J 1970, 118 (3), 355-65.
Li, S.; Van Den Diepstraten, C.; D'Souza, S. J.; Chan, B. M.; Pickering, J. G., Vascular smooth muscle cells orchestrate the assembly of type I collagen via alpha2betal integrin, RhoA, and fibronectin polymerization. Am J Pathol 2003, 163 (3), 1045-56.
Kadler, K. E.; Hojima, Y.; Prockop, D. J., Assembly of collagen fibrils de novo by cleavage of the type I pC-collagen with procollagen C-proteinase. Assay of critical concentration demonstrates that collagen self-assembly is a classical example of an entropy-driven process. J Biol Chem 1987, 262 (32), 15696-701.
Hynes, R. O.; Naba, A., Overview of the matrisome--an inventory of extracellular matrix constituents and functions. Cold Spring Harb Perspect Biol 2012, 4 (1), a004903.
Paten, J. A.; Martin, C. L.; Wanis, J. T.; Siadat, S. M.; Figueroa-Navedo, A. M.; Ruberti, J. W.; Deravi, L. F., Molecular Interactions between Collagen and Fibronectin: A Reciprocal Relationship that Regulates De Novo Fibrillogenesis. Chem 2019, 5 (8), 2126-2145.
K. W., D.; Kamocka, M. M.; McDonald, J. H., A practical guide to evaluating colocalization in biological microscopy. Am J Physiol Cell Physiol 2011, 300, C723-C742.
Williams, B. R.; Gelman, R. A.; Poppke, D. C.; Piez, K. A., Collagen fibril formation. Optimal in vitro conditions and preliminary kinetic results. J Biol Chem 1978, 253 (18), 6578-85.
Hayashi, T.; Nagai, Y., Factors affecting the interactions of collagen molecules as observed by in vitro fibril formation. I. Effects of small molecules, especially saccharides. J Biochem 1972, 72 (3), 749-58.
Wood, G. C.; Keech, M. K., The formation of fibrils from collagen solutions. 1. The effect of experimental conditions: kinetic and electron-microscope studies. Biochem J 1960, 75, 588-98.
Zemann, A.; Nguyen, D. T.; Bonn, G., Fast separation of underivatized carbohydrates by coelectroosmotic capillary electrophoresis. Electrophoresis 1997, 18 (7), 1142-1147.
Wang, X.; Xu, Y.; Fan, L.; Yong, Q.; Yu, S., Simultaneous Separation and Quantitative Determination of Monosaccharides, Uronic Acids, and Aldonic Acids By High Performance Anion-Exchange Chromatography Coupled With Pulsed Amperometric Detection in Corn Stover Prehydrolysates. BioResources 2012, 7 (4), 4614-4625.
Uquillas, J. A.; Akkus, 0., Modeling the Electromobility of Type-I Collagen Molecules in the Electrochemical Fabrication of Dense and Aligned Tissue Constructs. Ann Biomed Eng 2012, 40 (8), 1641-1653.
Kaye, G. I., Stereologic Measurement of Cell Volume Fraction of Rabbit Corneal Stroma. Archives of Ophthalmology 1969, 82 (6), 792-794.
Hayes, A. J.; MacPherson, S.; Morrison, H.; Dowthwaite, G.; Archer, C. W., The development of articular cartilage: evidence for an appositional growth mechanism. Anat Embryol (Berl) 2001, 203 (6), 469-79.
Hunziker, E. B.; Quinn, T. M.; Hauselmann, H. J., Quantitative structural organization of normal adult human articular cartilage. Osteoarthritis Cartilage 2002, 10 (7), 564-72.
Boos, N.; Weissbach, S.; Rohrbach, H.; Weiler, C.; Spratt, K. F.; Nerlich, A. G., Classification of age-related changes in lumbar intervertebral discs: 2002 Volvo Award in basic science. Spine (Phila Pa 1976) 2002, 27 (23), 2631-44.
Moore, M. J.; De Beaux, A., A quantitative ultrastructural study of rat tendon from birth to maturity. J Anat 1987, 153, 163-9.
McBride, D. J.; Hahn, R. A.; Silver, F. H., Morphological characterization of tendon development during chick embryogenesis: measurement of birefringence retardation. International Journal of Biological Macromolecules 1985, 7 (2), 71-76.
Kalson, N. S.; Lu, Y.; Taylor, S. H.; Starborg, T.; Holmes, D. F.; Kadler, K. E., A structure-based extracellular matrix expansion mechanism of fibrous tissue growth. Elife 2015, 4, e05958.
Nagy, I. Z.; Von Hahn, H. P.; Verzar, F., Age-related alterations in the cell nuclei and the DNA content of rat tail tendon. Gerontologia 1969, 15 (4), 258-64.
Ker, R. F.; Wang, X. T.; Pike, A. V., Fatigue quality of mammalian tendons. J Exp Biol 2000, 203 (Pt 8), 1317-27.
Wang, X. T.; Ker, R. F., Creep rupture of wallaby tail tendons. J Exp Biol 1995, 198 (Pt 3), 831-45.
Wang, X. T.; Ker, R. F.; Alexander, R. M., Fatigue rupture of wallaby tail tendons. J Exp Biol 1995, 198 (Pt 3), 847-52.
Gould, L.; Abadir, P.; Brem, H.; Carter, M.; Conner-Kerr, T.; Davidson, J.; DiPietro, L.; Falanga, V.; Fife, C.; Gardner, S.; Grice, E.; Harmon, J.; Hazzard, W. R.; High, K. P.; Houghton, P.; Jacobson, N.; Kirsner, R. S.; Kovacs, E. J.; Margolis, D.; McFarland Home, F.; Reed, M. J.; Sullivan, D. H.; Thom, S.; Tomic-Canic, M.; Walston, J.; Whitney, J. A.; Williams, J.; Zieman, S.; Schmader, K., Chronic wound repair and healing in older adults: current status and future research. J Am Geriatr Soc 2015, 63 (3), 427-38.
Li, Y.; Liu, Y.; Xia, W.; Lei, D.; Voorhees, J. J.; Fisher, G. J., Age-dependent alterations of decorin glycosaminoglycans in human skin. Sci Rep 2013, 3, 2422.
Ng, L.; Grodzinsky, A. J.; Patwari, P.; Sandy, J.; Plaas, A.; Ortiz, C., Individual cartilage aggrecan macromolecules and their constituent glycosaminoglycans visualized via atomic force microscopy. J Struct Biol 2003, 143 (3), 242-57.
Grande-Allen, K. J.; Calabro, A.; Gupta, V.; Wight, T. N.; Hascall, V. C.; Vesely, I., Glycosaminoglycans and proteoglycans in normal mitral valve leaflets and chordae: association with regions of tensile and compressive loading. Glycobiology 2004, 14 (7), 621-33.
Lee, H. Y.; Han, L.; Roughley, P. J.; Grodzinsky, A. J.; Ortiz, C., Age-related nanostructural and nanomechanical changes of individual human cartilage aggrecan monomers and their glycosaminoglycan side chains. J Struct Biol 2013, 181 (3), 264-73.
Markham, G. D.; Bock, C. L.; Bock, C. W., Hydration of the Carboxylate Group: An ab lnitio Molecular Orbital Study of Acetate-Water Complexes. J Struct Chem
Snowden, J. M.; Swann, D. A., Effects of Glycosaminoglycans and Proteoglycan on the in vitro Assembly and Thermal Stability of Collagen Fibrils. Biopolymers 1980, 19, 767-780.
Wen, X.; Mao, Z.; Han, Z.; Tuchin, V. V.; Zhu, D., In vivo skin optical clearing by glycerol solutions: mechanism. J Biophotonics 2010, 3 (1-2), 44-52.
Yeh, A. T.; Choi, B.; Nelson, J. S.; Tromberg, B. J., Reversible dissociation of collagen in tissues. J Invest Dermatol 2003, 121 (6), 1332-5.
Hovhannisyan, V.; Hu, P. S., Chen, S. J.; Kim, C. S.; Dong, C. Y., Elucidation of the mechanisms of optical clearing in collagen tissue with multiphoton imaging. J Biomed Opt 2013, 18 (4), 046004.
Hirshburg, J.; Choi, B.; Nelson, J. S.; Yeh, A. T., Correlation Between Collagen Solubility and Skin Optical Clearing Using Sugars. Lasers Surg Med 2007, 39, 140-144.
Comper, W. D.; Veis, A., Characterization of nuclei in in vitro collagen fibril formation. Biopolymers 1977, 16 (10), 2133-42.
Evans, C. H.; Drouven, B. J., The promotion of collagen polymerization by lanthanide and calcium ions. Biochem J 1983, 213 (3), 751-8.
Hijazi, K. M.; Singfield, K. L.; Veres, S. P., Ultrastructural response of tendon to excessive level or duration of tensile load supports that collagen fibrils are mechanically continuous. J Mech Behav Biomed Mater 2019, 97, 30-40.
Kohler, J.; Popov, C.; Klotz, B.; Alberton, P.; Prall, W. C.; Haasters, F.; Muller-Deubert, S.; Ebert, R.; Klein-Hitpass, L.; Jakob, F.; Schieker, M.; Docheva, D., Uncovering the cellular and molecular changes in tendon stem/progenitor cells attributed to tendon aging and degeneration. Aging Cell 2013, 12 (6), 988-99.
Birk, D. E.; Trelstad, R. L., Extracellular compartments in tendon morphogenesis: collagen fibril, bundle, and macroaggregate formation. J Cell Biol 1986, 103 (1), 231-40.
Richardson, S. H.; Starborg, T.; Lu, Y.; Humphries, S. M.; Meadows, R. S.; Kadler, K. E., Tendon development requires regulation of cell condensation and cell shape via cadherin-11-mediated cell-cell junctions. Mol Cell Biol 2007, 27 (17), 6218-28.
Moore, M. J.; De Beaux, A., A quantitative ultrastructural study of rat tendon from birth to maturity. J Anat 1987, 153, 163-9.
McBride, D. J.; Hahn, R. A.; Silver, F. H., Morphological characterization of tendon development during chick embryogenesis: measurement of birefringence retardation. International Journal of Biological Macromolecules 1985, 7 (2), 71-76.
Kalson, N. S.; Lu, Y.; Taylor, S. H.; Starborg, T.; Holmes, D. F.; Kadler, K. E., A structure-based extracellular matrix expansion mechanism of fibrous tissue growth. Elife 2015, 4, e05958.
Nagy, I. Z.; Von Hahn, H. P.; Verzar, F., Age-related alterations in the cell nuclei and the DNA content of rat tail tendon. Gerontologia 1969, 15 (4), 258-64.
Tuite, D. J.; Renstrom, P. A.; O′Brien, M., The aging tendon. Scand J Med Sci Sports 1997, 7 (2), 72-7.
Aukland, K.; Nicolaysen, G., Interstitial fluid volume: local regulatory mechanisms. Physiol Rev 1981, 61 (3), 556-643.
Aukland, K.; Reed, R. K., Interstitial-lymphatic mechanisms in the control of extracellular fluid volume. Physiol Rev 1993, 73 (1), 1-78.
Ryan, C. N.; Sorushanova, A.; Lomas, A. J.; Mullen, A. M.; Pandit, A.; Zeugolis, D. I., Glycosaminoglycans in Tendon Physiology, Pathophysiology, and Therapy. Bioconjug Chem 2015, 26 (7), 1237-51.
Riley, G. P.; Harrall, R. L.; Constant, C. R.; Chard, M. D.; Cawston, T. E.; Hazleman, B. L., Glycosaminoglycans of human rotator cuff tendons: changes with age and in chronic rotator cuff tendinitis. Ann Rheum Dis 1994, 53 (6), 367-76.
Ippolito, E.; Natali, P. G.; Postacchini, F.; Accinni, L.; De Martino, C., Morphological, immunochemical, and biochemical study of rabbit achilles tendon at various ages. J Bone Joint Surg Am 1980, 62 (4), 583-98.

1. A method of stabilizing a collagen solution against polymerization, the method comprising incubating an aqueous solution comprising soluble collagen monomers at a concentration of at least about 1 μg/mL and one or more saccharides at a concentration of at least about 0.01 M, wherein polymerization of said collagen monomers and/or collagen fibril formation in the solution is inhibited.

2. The method of claim 1, wherein the one or more saccharides are selected from the group consisting of galactose, iduronic acid, glucuronic acid, N-acetyl-galactosamine, N-acetyl-glucosamine, and combinations thereof.

3. The method of claim 1, wherein the total concentration of the one or more saccharides is greater than about 0.01 M, or greater than about 0.1 M, or greater than about 0.2 M, or greater than about 0.5 M, or greater than about 1 M.

4. The method of claim 1, wherein the solution comprises said collagen monomers at a concentration in the range from at least about 0.001 mg/mL to about 100 mg/mL.

5. The method of claim 4, wherein the collagen monomer concentration is at least about 0.5 mg/mL or at least about 1 mg/mL.

6. The method of claim 1, wherein the pH of the solution is in the range from about 4 to about 10.

7. The method of claim 1, wherein said incubating is carried out at a temperature in the range from about 0-40° C.

8. The method of claim 1, wherein the solution further comprises a negatively charged ionic species.

9. The method of claim 8, wherein the negatively charged ionic species is selected from the group consisting of acetate, formate, citrate, lactate, a C2-07 organic acid, or a combination thereof.

10. The method of claim 1, wherein the solution further comprises one or more glycosaminoglycans selected from the group consisting of keratin sulfate, dermatan sulfate, heparin sulfate, chondroitin sulfate, hyaluronic acid, and combinations thereof.

11. The method of claim 1, wherein the solution is devoid of any collagen solubility enhancers other than saccharides and glycosaminoglycans.

12. The method of claim 1, wherein the solution further comprises collagen dimers, trimers, oligomers, aggregates, fibrils, or a combination thereof.

13. The method of claim 1, wherein the solution is formulated for introduction into a body of a mammal, such as a human.

14. The method of claim 1, wherein the collagen in said solution does not spontaneously polymerize or form collagen fibrils when stored for a period of at least about one month.

15. The method of claim 1, whereby nucleation of collagen polymerization is inhibited in the solution compared to a solution lacking said saccharide.

16. A method of promoting collagenous tissue repair and/or remodeling in a mammalian subject in need thereof, the method comprising the steps of:
(a) providing an aqueous repair solution comprising soluble collagen monomers at a concentration of at least about 1 μg/mL and one or more saccharides at a concentration of at least about 0.01 M; and
(b) contacting the repair solution with a tissue repair and/or remodeling site in the subject, whereby collagen monomers from the solution polymerize, or form collagen fibrils, or incorporate into existing collagen fibrils at said repair and/or remodeling site in the subject.

17. The method of claim 16, wherein the one or more saccharides are selected from the group consisting of galactose, iduronic acid, glucuronic acid, N-acetyl-galactosamine, N-acetyl-glucosamine, and combinations thereof.

18. The method of claim 16, wherein the total concentration of the one or more saccharides is greater than about 0.01M, or greater than about 0.1 M, or greater than about 0.2 M, or greater than about 0.5 M, or greater than about 1 M.

19. The method of claim 16, wherein the repair solution comprises said collagen monomers at a concentration in the range from at least about 0.001 mg/mL to about 100 mg/mL.

20. The method of claim 19, wherein the collagen monomer concentration is at least about 0.5 mg/mL or at least about 1 mg/mL.

21. The method of claim 16, wherein the pH of the repair solution is in the range from about 4 to about 10.

22. The method of claim 16, wherein the repair solution further comprises a negatively charged ionic species.

23. The method of claim 22, wherein the negatively charged ionic species is selected from the group consisting of acetate, formate, citrate, lactate, a C₂-C₇ organic acid, or a combination thereof.

24. The method of claim 16, wherein the repair solution further comprises one or more glycosaminoglycans selected from the group consisting of keratin sulfate, dermatan sulfate, heparin sulfate, chondroitin sulfate, hyaluronic acid, and combinations thereof.

25. The method of claim 16, wherein the repair solution is devoid of any collagen solubility enhancers other than saccharides and glycosaminoglycans.

26. The method of claim 16, wherein the solution further comprises collagen dimers, trimers, oligomers, aggregates, fibrils, or a combination thereof.

27. The method of claim 16, which is performed in conjunction with a surgical procedure.

28. The method of claim 16, wherein said contacting comprises injection, infusion, continuous infusion, diffusion, dermal application, spraying, or surgical application or implantation of the repair solution at said tissue repair and/or remodeling site.

29. The method of claim 16, further comprising adjusting pH, ionic strength, collagen monomer concentration, and/or saccharide concentration of the repair solution.

30. The method of claim 16, wherein the method speeds repair and/or healing of a damaged connective tissue at said tissue repair and/or remodeling site.

31. The method of claim 16, wherein aids in treatment or repair in the subject of an injury, a wound, a bone fracture, a ruptured tendon, a ligament, a skin condition, or a damaged extracellular matrix.

32. The method of claim 16, wherein the tissue repair site comprises a wound, a broken or fractured bone, a ruptured tendon, an injured ligament, a skin lesion, a scar, a hernia, a damaged barrier membrane, an eye or a portion of an eye, an inflammation of a connective tissue, a site suspected of being subject to future injury or tissue damage, or a site suspected of sustaining an injury.

33. A tissue remodeling solution for use in the method of claim 16, the solution comprising soluble collagen monomers at a concentration of at least about 1 μg/mL and one or more saccharides at a concentration of at least about 0.01 M.

34. A medical device comprising the tissue remodeling solution of claim 31.

35. The medical device of claim 34 that is selected from the group consisting of a syringe, an implantable device, a wearable device, a wearable and removable device, an infusion device, a pump, and a catheter.

36. A tissue scaffold or artificial collagen-based tissue comprising the tissue remodeling solution of claim 33.

37. The tissue scaffold or artificial collagen-based tissue of claim 36 that is fabricated by a method comprising 3-D printing.

38. A kit comprising the device of claim 34 or 35 or the tissue scaffold or artificial collagen-based tissue of claim 36 or 37 and the tissue remodeling solution of claim 32.
